(19) United States
(12) Patent Application Publication
(10) Pub. No.: US 2015/0197494 A1
(43) Pub. Date: Jul. 16, 2015

(54) NO-RELEASING NITROOXY-METHYLENE-LINKED-COXIB CONJUGATES

(71) Applicant: Euclises Pharmaceuticals, Inc., St. Louis, MO (US)

(72) Inventors: John J. Talley, Saint Louis, MO (US); Eduardo J. Martinez, Bryn Mawr, PA (US); Kevin D. Jerome, St. Charles, MO (US)

(21) Appl. No.: 14/597,003

(22) Filed: Jan. 14, 2015

Related U.S. Application Data
(60) Provisional application No. 61/927,368, filed on Jan. 14, 2014.

(51) Int. Cl. C07D 231/12; A61K 45/06; A61K 39/395; A61K 31/415

(52) U.S. Cl. CPC C07D 231/12; A61K 31/415; A61K 45/06; A61K 39/3955

(57) ABSTRACT

The present invention provides NO-releasing nitrooxy-alkylene-linked-celecoxib conjugates, having the structure of Formula (I):

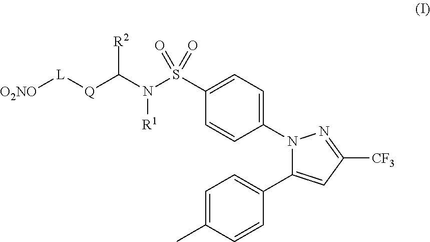

wherein R¹, R², Q, and L are as defined in the detailed description; pharmaceutical compositions comprising at least one compound of Formula (I); and methods useful for healing wounds, preventing and treating cancer, and treating actinic keratosis, cystic fibrosis, and acne, using a compound of Formula (I).

### CROSS-REFERENCE TO RELATED APPLICATIONS

This application claims the benefit of U.S. Provisional Application No. 61/927,368, filed on 14 Jan. 2014. The entire disclosure of the above application is incorporated herein by reference.

### FIELD

The present invention generally relates to NO-releasing coxib compounds, pharmaceutical compositions comprising the compounds, methods useful for treating a subject by administering a therapeutically effective amount of the compounds and methods for making the compounds. More specifically, the present invention relates to a class of NO-releasing nitrooxy-methylene-linked-coxib gastro-protective compounds, pharmaceutical compositions thereof and methods useful for healing wounds, preventing and treating cancer, and treating actinic keratosis, cystic fibrosis, and acne.

### BACKGROUND

Despite decades of effort, cancer remains an especially difficult disease for development of therapeutics. According to the Cancer Prevention Coalition (University of Illinois), cancer rates have increased 24% in the past thirty years even after adjusting for aging of the population. Remarkably, despite significant progress during this period, the overall five-year survival rates have remained virtually static (approximately 50% depending on the cancer). Thus, new drugs are required to develop more effective life-saving cancer therapies.

Celecoxib, a selective COX-2 inhibitor, is one of the world's most successful drugs, alleviating pain and inflammation for millions of patients. In addition, COX-2 over-expression has been found in several types of human cancers, such as colon, breast, lung, prostate, and pancreas, and appears to control many cellular processes. COX-2 plays a role in carcinogenesis, apoptosis, and angiogenesis and, therefore, represents an excellent drug target for the development of novel medicines for prevention and/or treatment of human cancers. Currently, celecoxib is approved for limited use in the reduction of polyps in familial adenomatous polyposis (FAP).

The Adenoma Prevention with Celecoxib (APC) trial demonstrated human efficacy of celecoxib in the prevention of sporadic colorectal adenoma. However, this trial also showed that the elevated dose of celecoxib required for anti-cancer efficacy was accompanied by concomitant increase in adverse cardiovascular (CV) events (Cancer Prev. Res. 2, 310-321(2009)).

Development of more potent or selective COX-2 inhibitors does not improve CV safety; this liability is thought to be a mechanism-based effect. This was demonstrated in the VIGOR trial by Vioxx®, an extremely potent and highly selective COX-2 inhibitor withdrawn from the market in 2004 due to CV concerns about increased risk of heart attack and stroke with long term, high dose use. These facts have undermined the development of novel COX-2 inhibitors and slowed research to expand their utility to other disease indications, such as cancer.

Nitric oxide (NO) is an important endogenous signaling molecule and vasodilator. NO is synthesized from L-arginine by the enzyme NO synthase (NOS), which exists in three distinct isoforms, namely, the constitutively expressed endothelial (eNOS) and neuronal (nNOS) forms, and the mainly inducible form (iNOS). Arginine administration has been shown to reduce blood pressure and renal vascular resistance in essential hypertensive patients with normal or insufficient renal function (Am. J. Hypertens. 12, 8-15 (1999)). It has also been shown that NO deficiency promotes vascular side-effects of celecoxib and other COX inhibitors (Blood 108, 4059-4062 (2006)).

The role of NO in cancer is complex; however, pharmacological evidence using NO-releasing compounds of NSAID's has shown increased anti-tumor efficacy in cell culture and animal cancer models. The different molecular mechanisms of NO are expected to simultaneously enhance anti-cancer efficacy of celecoxib and improve CV safety by preventing an increase in blood pressure associated with COX-2 inhibition, while maintaining gastric-sparing properties superior to NSAID's.

Diverse molecular mechanisms of NO delivery are well known. For example, it is reported that nitric oxide-donating NSAID's (NO-sulindac, NO-ibuprofen, NO-indomethacin, and NO-aspirin) inhibit the growth of various cultured human cancer cells, providing evidence of a tissue type-independent effect (J. Pharmacol. Exp. Ther. 303, 1273-1282 (2002)).

In another example, it is reported that nitric oxide-donating aspirin prevented pancreatic cancer in a hamster tumor model (Cancer Res. 66, 4503-4511 (2006)).

Two isoforms of cyclooxygenase (COX) are known to exist, a constitutive form (COX-1) present in nearly all tissues and an inducible form (COX-2) upregulated in response to inflammatory stimuli. The discovery of COX-2 led to the development of selective COX-2 inhibitors as anti-inflammatory drugs (coxibs), which were shown to be largely devoid of the antiplatelet activity and gastrointestinal ulcerogenicity believed to be associated with inhibition of COX-1.

NSAID's are among the most widely used treatments for pain, fever, and inflammation, and have long been known to reduce the risk of cancer in multiple organ sites. The use of aspirin in treatment and prevention of cancer has wide-spread support in the medical community; however, the risks of regular aspirin use are also well established and the risk-benefit profile is not sufficient to recommend aspirin treatment for cancer prevention. With the advent of coxibs, research has focused on COX-2 as a target for the treatment and prevention of certain cancers. Compelling data from the APC trial, described above, demonstrated that celecoxib was useful in preventing sporadic colorectal adenoma in patients at high risk for colorectal cancer.

Lung cancer is the leading cause of cancer-related deaths in the US and is responsible for more deaths than breast, prostate, and colon cancers combined. Current research suggests that COX-2 and epidermal growth factor receptor (EGFR) are important mediators in non-small cell lung cancer (NSCLC). One study demonstrates a strong cooperative effect on slowing tumor progression by blocking both the EGFR and COX-2 pathways using gefitinib and celecoxib (Zhang, X, Clin. Cancer Res.11, 6261-6269 (2005)).

In human NSCLC patients, a combination of erlotinib (a tyrosine kinase inhibitor) and celecoxib showed high response rates, and demonstrable clinical benefit (Reckamp, K. L, Clin. Cancer Res. 12, 3381-3388 (2006)). NSCLC currently represents one of the preferred indications for COX-2 inhibition cancer therapy (Brown, J. R., Clin. Cancer Res. 10, 4266s-4269s (2004); and Gadgeel, S. M., Cancer 110, 2775-2784 (2007)).

A key feature of COX-2 biology is its ability alone to cause cancer formation in a number of transgenic mouse models. COX-2 derived PGE2 plays a prominent role in tumor growth and is the most abundant prostanoid in many human malignancies. Metabolism of arachidonic acid by COX-2 leads to the formation of several prostaglandins (PGs) that bind to tumor suppressor p53, preventing p53-mediated apoptosis. COX-2-derived PGE2 promotes epithelial-to-mesenchymal transition and, thus, increases resistance to EGFR tyrosine kinase inhibitors in lung cancer (Krysan, K., J. Thorac. Oncol. 3, 107-110 (2008)).

Colorectal cancer (CRC) is the second-leading cause of cancer-related deaths in the US. Colorectal cancer progression and metastasis occurs through aberrant signaling through the prostaglandin-endoperoxide synthase 2 (PTGS2) and epithelial growth factor (EGF) signaling pathways (Wang, D., Cancers 3, 3894-3908 (2011)). COX-2 over-expression contributes to PTGS2 signaling and therefore COX-2 inhibitors may provide a successful treatment modality for colorectal neoplasia (Eberhart, C. E., Gastroenterology 107, 1183-1188 (1994)).

Nitric oxide exhibits a number of important pharmacological actions including vascular relaxation (vasodilatation) and inhibition of platelet aggregation and adhesion. Inhibition of NO synthesis leads to an increase in systemic blood pressure. NO also prevents atherogenesis by inhibiting vascular smooth muscle cell proliferation, and preventing low-density lipoprotein oxidation and macrophage activation. Vascular NO generation is important in controlling blood pressure, and a growing body of evidence indicates that NO signaling is a key factor in counteracting the onset and development of several CV diseases including hypertension, myocardial infarction, and stroke. NO can be used to counteract CV liabilities associated with COX-2 inhibition.

NO-releasing COX inhibitors were originally created to improve gastrointestinal (GI) tolerability (Inflammopharmacology 11(4), 415-22 (2003)). Naproxcinod is a NO-releasing pro-drug of the NSAID naproxen. Naproxcinod showed significantly improved GI tolerability compared to naproxen alone in a chronic rat study (Life Sciences 62, 235-240 (1998)). In another example, L-arginine, coadministered with the NSAID ibuprofen, showed a protective effect on gastric mucosa against ibuprofen-induced mucosal lesions (Free Radic. Res. 38(9), 903-11 (2004)).

NO modulates the activity of transcription factor NF-κB, which represents a potential mechanism for inflammation control, but also regulation of apoptotic mechanisms. NO promotes apoptosis and can reverse tumor cell resistance to chemotherapeutic agents. Studies with NO-releasing NSAID's have shown that NO contributes to anti-cancer activity in cell culture and enhanced in vivo efficacy in rodent cancer models. For example, it is reported that nitric oxide-naproxen is an effective agent against carcinogenesis in rodent models of colon and urinary bladder cancers (Cancer Prev. Res. 2, 951-956 (2009)).

Nitric oxide-releasing prodrugs useful in the treatment of inflammation and the reduction of adverse cardiovascular events associated with high doses of anti-inflammatories are reported in U.S. Pat. No. 7,932,294. The compounds described therein include celecoxib substituted with a nitrooxy-ethylene-disulfide-ethyleneoxy-carbonyl radical to sulfonamide nitrogen, yielding structure (1) below:

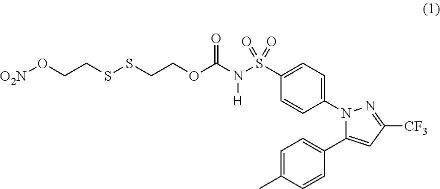 [SMILES: Cc1ccc(-c2cc(C(F)(F)F)nn2-c2ccc(S(=O)(=O)NC(=O)OCCSSCCO[N+](=O)[O-])cc2)cc1]

Nitrate prodrugs useful in the treatment of inflammatory, ischemic, degenerative and proliferative diseases are reported in EP 01336602. The compounds described therein include celecoxib substituted with nitrooxy-alkylene-carbony or a carboxy(dinitrooxy)ethylene-carbonyl radical to sulfonamide nitrogen yielding, respectively, structures (2*) & (3) below:

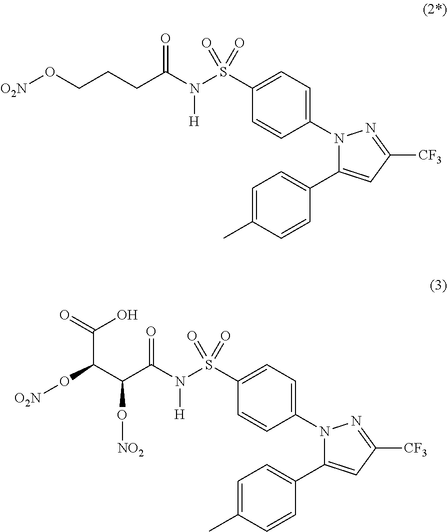 [SMILES: Cc1ccc(-c2cc(C(F)(F)F)nn2-c2ccc(S(=O)(=O)NC(=O)CCCO[N+](=O)[O-])cc2)cc1 ; Cc1ccc(-c2cc(C(F)(F)F)nn2-c2ccc(S(=O)(=O)NC(=O)[C@@H](O[N+](=O)[O-])[C@@H](O[N+](=O)[O-])C(=O)O)cc2)cc1]

*Note: Structure (2*) above is also reported in U.S. Pat. No. 7,776,902 and WO 2004/000781.

Nitric oxide-releasing compounds useful in the treatment of COX-2 mediated diseases and cancer are reported in WO 2004/037798. The compounds described therein include celecoxib substituted with nitrooxy-alkylene-carbonyl or a nitrooxy-butylene-O-carbonyl radical at sulfonamide nitrogen, yielding, respectively, structures (4) & (5) below:

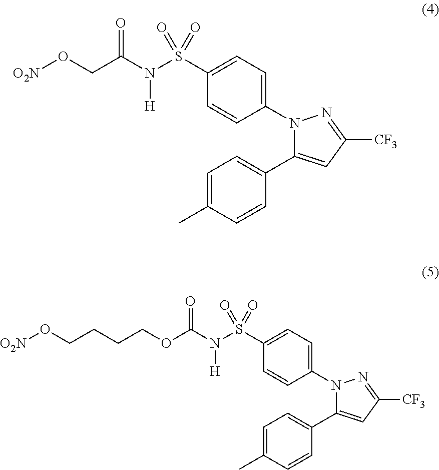 [SMILES: Cc1ccc(-c2cc(C(F)(F)F)nn2-c2ccc(S(=O)(=O)NC(=O)CO[N+](=O)[O-])cc2)cc1 ; Cc1ccc(-c2cc(C(F)(F)F)nn2-c2ccc(S(=O)(=O)NC(=O)OCCCCO[N+](=O)[O-])cc2)cc1]

### SUMMARY OF THE INVENTION

Herein described is a family of NO-releasing coxib conjugates which provides a therapeutic benefit to a subject with a disease indication, such as cancer, actinic keratosis, cystic fibrosis, or acne, or provides a wound healing benefit to a subject. Such NO-releasing coxib conjugates can reduce gastric erosion of cancer therapy, improve CV safety, permit higher dose of cancer-treating compound, enhance cancer-treating efficacy, and/or maintain gastric-sparing properties superior to NSAID's.

In another aspect, there is provided a compound of Formula (I):

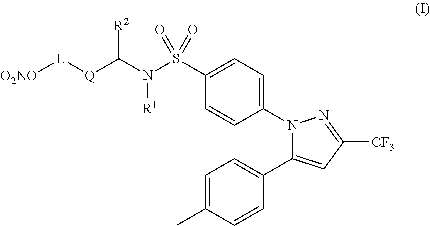

and pharmaceutically acceptable salts thereof wherein R¹, R², Q, and L are as defined in the detailed description.

Compounds of the present invention can exist in tautomeric, geometric or stereoisomeric forms. Ester, metabolite, oxime, prodrug, onium, hydrate, solvate, and N-oxide forms of a compound of Formula (I) are also embraced by the invention. The present invention considers all such compounds, including, but not limited to, cis- and trans-geometric isomers (Z- and E-geometric isomers), R- and S-enantiomers, diastereomers, d-isomers, l-isomers, atropisomers, epimers, conformers, rotamers, mixtures of isomers, and racemates thereof, as falling within the scope of the invention.

### DETAILED DESCRIPTION

### A. Compounds

The present invention provides compounds, or pharmaceutically acceptable salts,

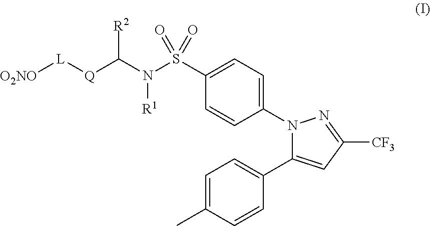

wherein R¹ is selected from the group consisting of acyl, arylcarbonyl, heteroarylcarbonyl, carboxyalkylenecarbonyl, and alkyl-O-carbonylalkylenecarbonyl; R² is selected from the group consisting of H, alkyl, cycloalkyl, aryl, aralkyl, and heterocyclyl; -Q-is selected from the group consisting of O,

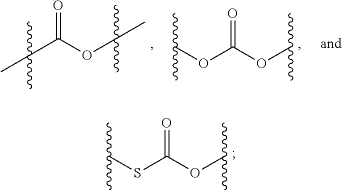 [SMILES: *OC(*)=O ; *OC(=O)O* ; *OC(=O)S*]

-L- is C₁₋₈ alkylene, wherein one, two, or three —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂; R³ is independently selected from the group consisting of H, alkyl, aryl, aralkyl, heterocyclyl, halogen, carboxy, carboxyalkylene, acyl, and nitrooxy C₁₋₃ alkylene; and R⁴ is selected from the group consisting of alkyl, aryl, aralkyl, heterocyclyl, carboxyalkylene, and acyl.

The present invention is also directed to a subclass of compounds, including pharmaceutically acceptable salts of compounds, wherein compounds have the structure of Formula (II):

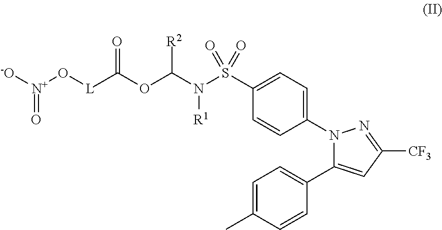

wherein R¹ is acyl; R² is selected from the group consisting of H, alkyl, and cycloalkyl; -L- is C₁₋₆ alkylene, wherein one or two —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂; R³ is independently selected from the group consisting of H, alkyl, aryl, aralkyl, heterocyclyl, halogen, carboxy, carboxyalkylene, acyl, and nitrooxy C₁₋₃ alkylene; and R⁴ is selected from the group consisting of alkyl, aryl, aralkyl, heterocyclyl, carboxyalkylene, and acyl.

In another family of the compounds of Formula (II), R² is selected from the group consisting of H and alkyl; -L- is C₁₋₆ alkylene, wherein one —CH₂— radical may be optionally replaced with —CH(R³)—; and R³ is selected from the group consisting of H, alkyl, and aryl.

In another family of the compounds of Formula (I), R¹ is acetyl; R² is H or methyl; -L- is C₁₋₄ alkylene, wherein one —CH₂— radical may be optionally replaced with —CH(R³)—; and R³ is selected from the group consisting of H, methyl, or phenyl. Non-limiting examples include:

| Ex. | Structure | Compound Name |
|---|---|---|
| 1 | 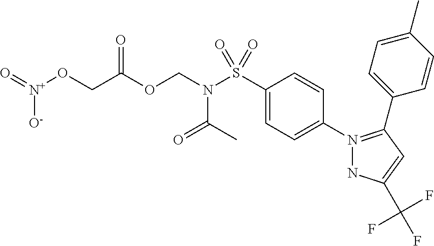 | (N-((4-(5-(p-tolyl)-3-(trifluoromethyl)-1H- pyrazol-1- yl)phenyl)sulfonyl)acetamido)methyl 2- (nitrooxy)acetate |
| 2 | 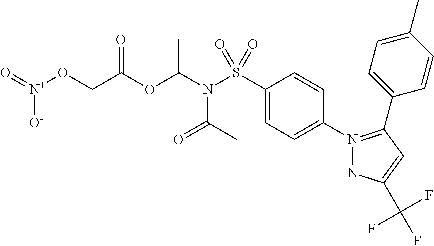 | 1-(N-((4-(5-(p-tolyl)-3-(trifluoromethyl)-1H- pyrazol-1-yl)phenyl)sulfonyl)acetamido)ethyl 2-(nitrooxy)acetate |
| 3 | 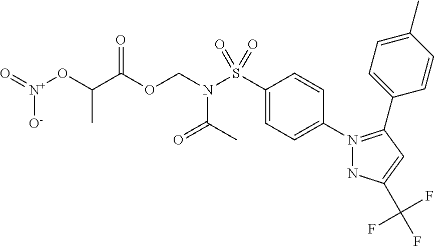 | (N-((4-(5-(p-tolyl)-3-(trifluoromethyl)-1H- pyrazol-1- yl)phenyl)sulfonyl)acetamido)methyl 2- (nitrooxy)propanoate |
| 4 | 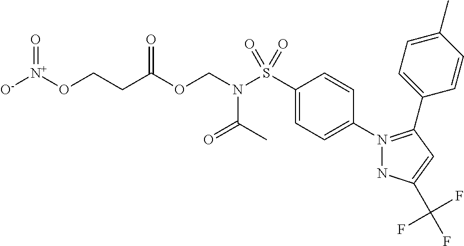 | (N-((4-(5-(p-tolyl)-3-(trifluoromethyl)-1H- pyrazol-1- yl)phenyl)sulfonyl)acetamido)methyl 3- (nitrooxy)propanoate |
| 5 | 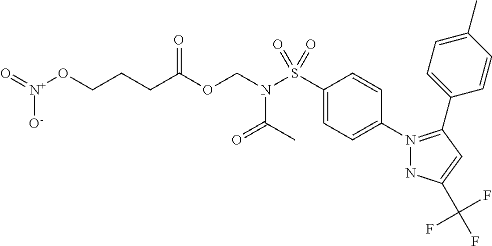 | (N-((4-(5-(p-tolyl)-3-(trifluoromethyl)-1H- pyrazol-1- yl)phenyl)sulfonyl)acetamido)methyl 4- (nitrooxy)butanoate |
| 6 | 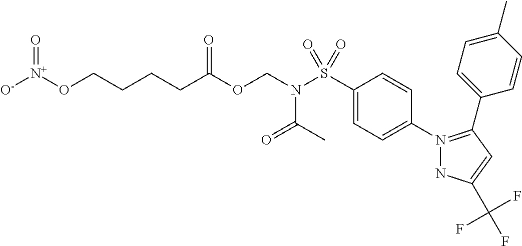 | (N-((4-(5-(p-tolyl)-3-(trifluoromethyl)-1H- pyrazol-1- yl)phenyl)sulfonyl)acetamido)methyl 5- (nitrooxy)pentanoate |
| 7 | 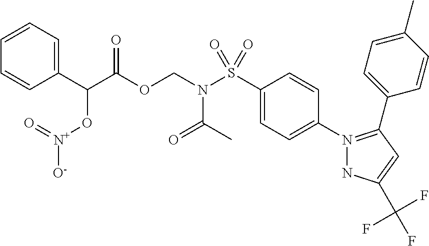 | (N-((4-(5-(p-tolyl)-3-(trifluoromethyl)-1H- pyrazol-1- yl)phenyl)sulfonyl)acetamido)methyl 2- (nitrooxy)-2-phenylacetate |

The present invention is also directed to a subclass of compounds, including pharmaceutically acceptable salts of compounds, wherein compounds have the structure of Formula (III):

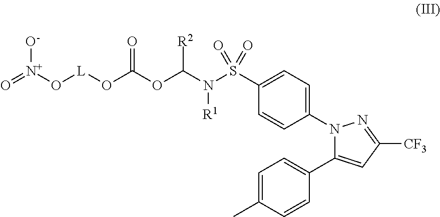

wherein R¹ is acyl; R² is selected from the group consisting of H, alkyl, and cycloalkyl; -L- is C₂₋₆ alkylene, wherein one or two —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂; R³ is independently selected from the group consisting of H, alkyl, aryl, aralkyl, heterocyclyl, halogen, carboxy, carboxyalkylene, acyl, and nitrooxy C₁₋₃ alkylene; and R⁴ is selected from the group consisting of alkyl, aryl, aralkyl, heterocyclyl, carboxyalkylene, and acyl.

In another family of the compounds of Formula (III), R² is selected from the group consisting of H and alkyl; -L- is C₂₋₆ alkylene, wherein one —CH₂— radical may be optionally replaced with —CH(R³)—; and R³ is selected from the group consisting of H, alkyl, aryl, and nitrooxy C₁₋₃ alkylene.

In another family of the compounds of Formula (III), R¹ is acetyl; R² is H or methyl; -L- is C₂₋₄ alkylene, wherein one —CH₂— radical may be optionally replaced with —CH(R³)—; and R³ is selected from the group consisting of H, methyl, or nitrooxymethylene. Non-limiting examples include:

| Ex. | Structure | Compound Name |
|---|---|---|
| 8 | 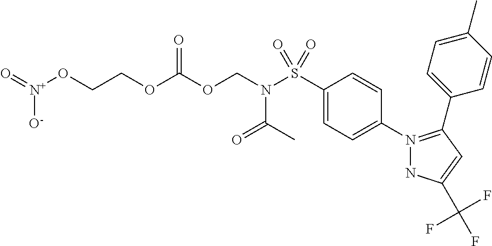 | 2-(nitrooxy)ethyl ((N-((4-(5-(mtolyl)-3- (trifluoromethyl)-1H-pyrazol-1- yl)phenyl)sulfonyl)acetamido)methyl) carbonate |
| 9 | 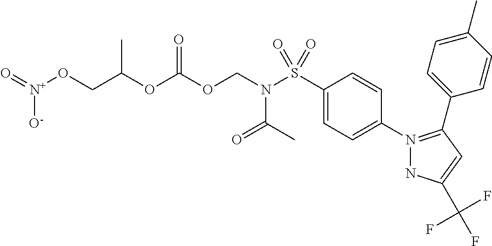 | 1-(nitrooxy)propan-2-yl ((N-((4-(5-(p-tolyl)-3- (trifluoromethyl)-1H-pyrazol-1- yl)phenyl)sulfonyl)acetamido)methyl) carbonate |
| 10 | 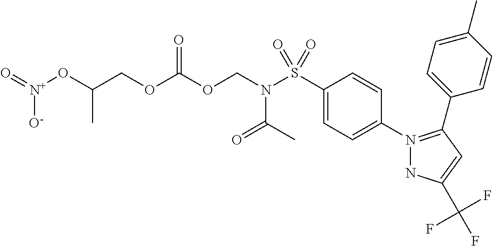 | 2-(nitrooxy)propyl ((N-((4-(5-(p-tolyl)-3- (trifluoromethyl)-1H-pyrazol-1- yl)phenyl)sulfonyl)acetamido)methyl) carbonate |
| 11 | 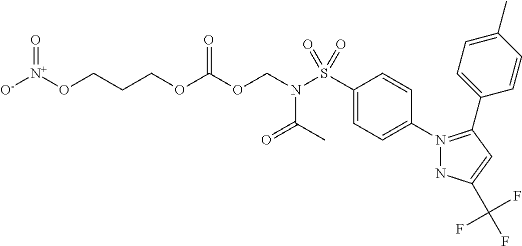 | 3-(nitrooxy)propyl ((N-((4-(5-(p-tolyl)-3- (trifluoromethyl)-1H-pyrazol-1- yl)phenyl)sulfonyl)acetamido)methyl) carbonate |
| 12 | 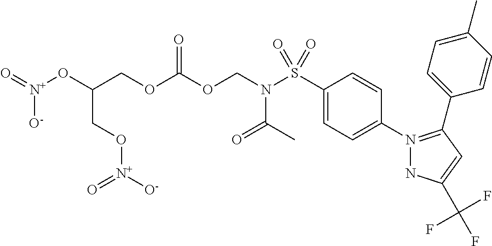 | 2,3-bis(nitrooxy)propyl ((N-((4-(5-(p-tolyl)-3- (trifluoromethyl)-1H-pyrazol-1- yl)phenyl)sulfonyl)acetamido)methyl) carbonate |
| 13 | 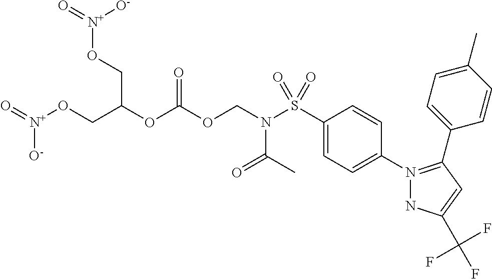 | 1,3-bis(nitrooxy)propan-2-yl ((N-((4-(5-(p- tolyl)-3-(trifluoromethyl)-1H-pyrazol-1- yl)phenyl)sulfonyl)acetamido)methyl) carbonate |
| 14 | 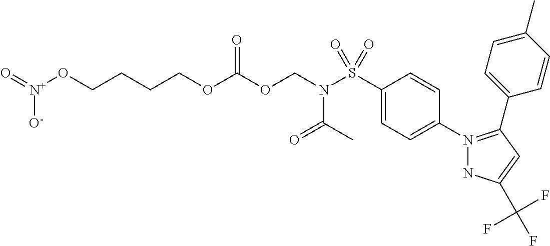 | 4-(nitrooxy)butyl ((N-((4-(5-(mtolyl)-3- (trifluoromethyl)-1H-pyrazol-1- yl)phenyl)sulfonyl)acetamido)methyl) carbonate |

The present invention is also directed to a subclass of compounds, including pharmaceutically acceptable salts of compounds, wherein compounds have the structure of Formula (IV):

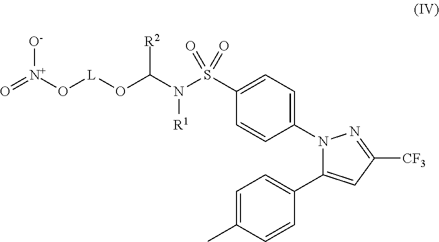

wherein R¹ is acyl; R² is selected from the group consisting of H, alkyl, and cycloalkyl; -L- is C₂₋₆ alkylene, wherein one or two —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂; R³ is independently selected from the group consisting of H, alkyl, aryl, aralkyl, heterocyclyl, halogen, carboxy, carboxyalkylene, acyl, and nitrooxy C₁₋₃ alkylene; and R⁴ is selected from the group consisting of alkyl, aryl, aralkyl, heterocyclyl, carboxyalkylene, and acyl.

In another family of the compounds of Formula (IV), R² is selected from the group consisting of H and alkyl; -L- is C₂₋₆ alkylene, wherein one —CH₂— radical may be optionally replaced with —CH(R³)—; and R³ is selected from the group consisting of H, alkyl, and nitrooxy C₁₋₃ alkylene.

In another family of the compounds of Formula (IV), R¹ is acetyl; R² is H or methyl; -L- is C₂₋₄ alkylene; and R³ is H. Non-limiting examples include:

| Ex. | Structure | Compound Name |
|---|---|---|
| 15 | 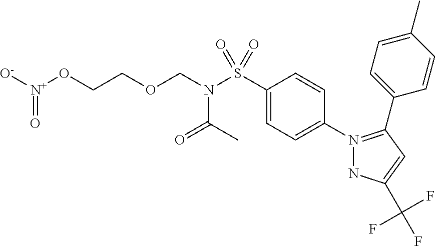 | 2-((N-((4-(5-(p-tolyl)-3-(trifluoromethyl)-1H- pyrazol-1- yl)phenyl)sulfonyl)acetamido)methoxy)ethyl nitrate |
| 16 | 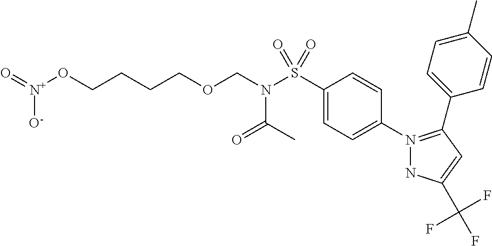 | 4-((N-((4-(5-(p-tolyl)-3-(trifluoromethyl)-1H- pyrazol-1- yl)phenyl)sulfonyl)acetamido)methoxy)butyl nitrate |

### B. Other Embodiments

In another embodiment, there is provided a pharmaceutical composition comprising a compound of the structural formulae herein, and a pharmaceutically-acceptable carrier.

In another embodiment, the pharmaceutical composition further comprises one or more additional pharmaceutically active compounds.

In another embodiment, there is provided a method for treating or preventing a disease condition comprising administering to a subject a therapeutically effective amount of a compound of the structural formulae herein, wherein the condition to be treated or prevented includes, for example, cancer. Further non-limiting examples include non-small cell lung cancer, skin cancer, liver cancer, colorectal cancer (including metastatic colorectal cancer, and FAP), glioblastoma (and other CNS related cancers), squamous cell cancer, bladder cancer, breast cancer, biliary tract cancer, cervical cancer, prostate cancer, small cell lung cancer, ovarian cancer, pancreatic cancer, gastrointestinal cancer, and CNS cancer.

In another embodiment, there is provided a method for healing wounds, comprising administering to a subject a therapeutically effective amount of a compound of the structural formulae herein.

In another embodiment, there is provided a method for treating a condition, comprising administering to a subject a therapeutically effective amount of a compound of the structural formulae herein, wherein the condition to be treated includes, for example, actinic keratosis, cystic fibrosis, and/or acne.

In another embodiment, there is provided a method for treating a condition comprising administering to a subject a therapeutically effective amount of a compound of the structural formulae herein, wherein the condition to be treated includes, for example, autoimmune disorder, inflammatory disorder, and/or auto-inflammatory disorder.

In another embodiment, there is provided a method that comprises administering a combination of a compound of the structural formulae herein, and at least one additional pharmaceutically active compound.

In another embodiment, there is provided a use of a compound of the structural formulae herein for manufacture of a medicament for treatment of a disease condition in a subject.

In another embodiment, there is provided a method for preparing a compound of the structural formulae herein.

In another embodiment, there is provided an intermediate useful in making a compound of the structural formulae herein.

In another embodiment, there is provided a method of enhancing cancer-treating efficacy by activating both NO and COX-2-inihibitor anti-tumor mechanisms in a subject, by administering a therapeutically effective amount of a compound of the structural formulae herein.

In another embodiment, there is provided a method of treating a subject suffering from a disease condition caused by COX-2 over-expression, including but not limited to cancer, by administering a therapeutically effective amount of a compound of the structural formulae herein.

In another embodiment, there is provided a method of improving CV safety in a subject, by administering a therapeutically effective amount of a compound of the structural formulae herein.

In another embodiment, there is provided a method of treating a subject suffering from a disease condition, including but not limited to cancer, by administering a high dose of a compound of the structural formulae herein.

In another embodiment, there is provided a method of gastro-protection in a subject, comprising administering a therapeutically effective amount of a compound of the structural formulae herein.

In another embodiment, there is provided a method of releasing NO in a subject, comprising administering a therapeutically effective amount of a compound of the structural formulae herein.

In another embodiment, there is provided a method of gastro-protection in a subject, comprising administering a therapeutically effective amount of a compound of the structural formulae herein, which releases NO in the subject, preferably by sustained release.

In another embodiment, there is provided a method of gastro-protection in a subject, comprising administering a therapeutically effective amount of a compound of the structural formulae herein, which releases NO in the subject, preferably by sustained release, wherein the NO release is likely caused by an enzymatic mechanism acting on the NONOate moiety of the compound of the structural formulae herein.

In another embodiment, there is provided a method of gastro-protection in a subject, comprising administering a therapeutically effective amount of a compound of the structural formulae herein, which releases NO in the subject, preferably by sustained release, wherein the NO release is likely caused by a non-enzymatic mechanism acting on the NONOate moiety of the compound of the structural formulae herein.

In another embodiment, there is provided a method of treating a subject suffering from a disease condition, including but not limited to cancer, comprising administering a therapeutically effective amount of a compound of the structural formulae herein, without causing substantial adverse, cardiovascular events.

In another embodiment, there is provided a method of treating a subject suffering from a disease condition, including but not limited to cancer, comprising administering a therapeutically effective amount of a compound of the structural formulae herein, without causing substantial changes in blood pressure, while maintaining gastric-sparing properties.

It will be recognized that the compounds of this invention can exist in radiolabeled form, i.e., the compounds may contain one or more atoms containing an atomic mass or mass number different from the atomic mass or mass number usually found in nature (e.g., an isotope). Alternatively, a plurality of molecules of a single structure may include at least one atom that occurs in an isotopic ratio that is different from the isotopic ratio found in nature. Radioisotopes of hydrogen, carbon, phosphorous, fluorine, chlorine and iodine include ²H, ³H, ¹¹C, ¹³C, ¹⁴C, ¹⁵N, ³⁵S, ¹⁸F, ³⁶Cl, ¹²⁵I, ¹²⁴I, and ¹³¹I respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Persons skilled in the art recognize that deuterium has been used to improve metabolic stability of compounds, and that principle can be applied to these compounds. Compounds that contain isotopes 11C, ¹³N, ¹⁵O, ¹²⁴I and is a ¹⁸F are well suited for positron emission tomography. Radiolabeled compounds of the structural formulae herein and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

### C. General Synthetic Schemes

Compounds of the present invention can be prepared using methods illustrated in general synthetic schemes and experimental procedures detailed below. These general synthetic schemes and experimental procedures are presented for purposes of illustration and are not intended to be limiting. Starting materials used to prepare compounds of the present invention are commercially available or can be prepared using routine methods known in the art.

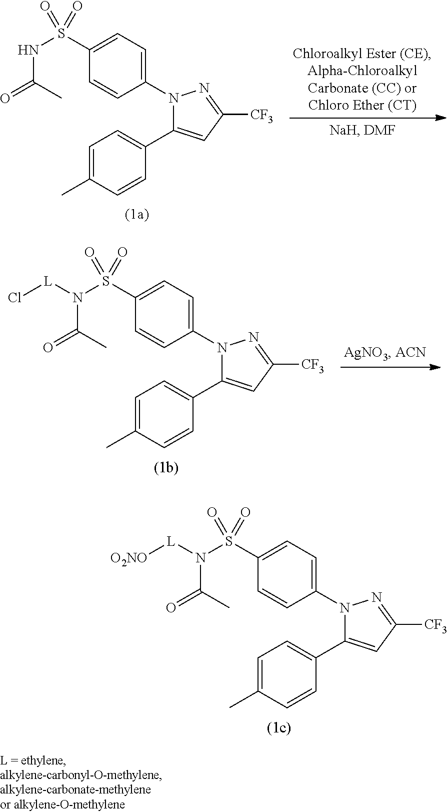

N-Acetylcelecoxib (1a) is prepared according to WO 1997/38986.

Alpha-Chloroalkyl Ester (CE) Formation (See also Ulich, L. H., et. al., J. Am. Chem. Soc. 1921, 43, 660-667): An acyl chloride (1.0 eq.; See Table 1) and an aldehyde (1.2 eq.; See Table 1) are mixed in a round-bottomed flask equipped with a reflux water condenser. A minute quantity of anhydrous zinc chloride is added resulting in considerable heat evolution (in many cases causing the reaction mixture to boil gently). The reaction is heated further to 90° C. for 3 to 4 hrs, and at the end of this time the reaction mixture is distilled with a fractionating column preferably under diminished pressure. The distillate is then redistilled either under atmospheric or diminished pressure to yield halogenated methyl ester (CE). Suitable aldehydes include, for example, paraformaldehyde, paraldehyde, etc. (See Table 1).

TABLE 1
Starting Materials and Resulting Chloroalkyl Esters (CE)

| Acyl Chloride & Aldehyde | CAS# | (CE) Product | No. |
|---|---|---|---|
| Bromoacetyl chloride & paraformaldehyde | [22118-09-8] & [30525-89-4] | 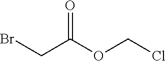 | CE1 |
| Bromoacetyl chloride & paraldehyde | [22118-09-8] & [123-63-7] | 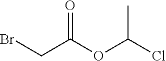 | CE2 |
| 2-Bromopropionyl chloride & paraformaldehyde | [7148-74-5] & [30525-89-4] | 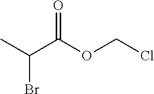 | CE3 |
| 3-Bromopropionyl chloride & paraformaldehyde | [15486-96-1] & [30525-89-4] | 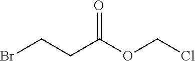 | CE4 |
| 4-Bromobutyryl chloride & paraformaldehyde | [927-58-2] & [30525-89-4] | 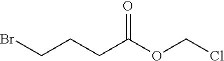 | CE5 |
| 5-Bromovaleryl chloride & paraformaldehyde | [4509-90-4] & [30525-89-4] | 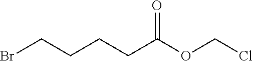 | CE6 |
| 2-Chloro-2-phenylacetyl chloride & paraformaldehyde | [2912-62-1] & [30525-89-4] | 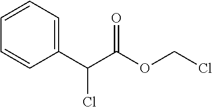 [SMILES: O=C(OCCl)C(Cl)c1ccccc1] | CE7 |

Alpha-Chloroalkyl Carbonate (CC) Formation: Chloromethyl chloroformate (1.0 eq) (or 1-chloroethyl chloroformate in the case of CC8, See Table 2) is dissolved in tetrahydrofuran (THF) (1.0 M) and cooled to 0° C. An alcohol (See Table 2) is added (1.0 eq) followed by triethylamine (1.0 eq). Triethylammonium chloride precipitate forms as the reaction proceeds. After 3-5 hrs, one volume of heptanes is added, and the mixture is cooled to −10° C. using a brine ice bath to further precipitate the ammonium salt. The mixture is filtered and washed with cold 1:1 heptanes-THF. The filtrate is evaporated under reduced pressure to afford (CC) and used directly in the next reaction. In addition to chloromethyl chloroformate, other chloroformates such as 1-chloroethyl chloroformate and 1-chloro-2-methylpropyl chloroformate are used to make additional analogs.

TABLE 2
Starting Materials and Resulting Chloroalkyl Carbonates (CC)

| Alcohol & Chloroalkyl chloroformate | CAS # | (CC) Products | ID. No. |
|---|---|---|---|
| 2-Bromoethanol & Chloromethyl chloroformate | [540-51-2] & [22128-62-7] | 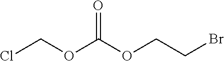 | CC1 |
| 1-Bromo-2-propanol & Chloromethyl chloroformate | [19686-73-8] & [22128-62-7] | 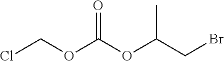 | CC2 |
| 2-Bromo-1-propanol & Chloromethyl chloroformate | [598-18-5] & [22128-62-7] | 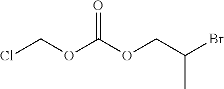 | CC3 |
| 3-Bromo-1-propanol & Chloromethyl chloroformate | [627-18-9] & [22128-62-7] | 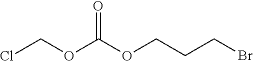 | CC4 |
| 2,3-Dibromo-1-propanol & Chloromethyl chloroformate | [96-13-9] & [22128-62-7] | 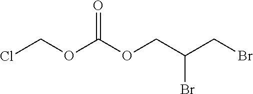 [SMILES: O=C(OCCl)OCC(Br)CBr] | CC5 |
| 1,3-Dibromo-2-propanol& Chloromethyl chloroformate | [96-21-9] & [22128-62-7] | 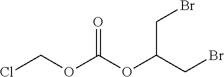 [SMILES: O=C(OCCl)OC(CBr)CBr] | CC6 |
| 4-Bromo-1-butanol & Chloromethyl chloroformate | [33036-62-3] & [22128-62-7] | 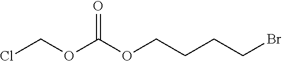 [SMILES: O=C(OCCl)OCCCCBr] | CC7 |

TABLE 3
Chloroalkyl Ethers (CT)

| CAS # or Reference | | |
|---|---|---|
| to Make | (CT) Structures | ID. No. |
| [1462-35-7] | 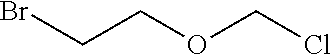 | CT1 |
| U.S. Pat. No. 3,995,094 | 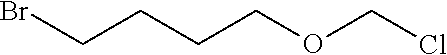 | CT2 |

Alkylation of sulfonamide: N-Acetylcelecoxib (1a) (1.0 eq) is dissolved in N,N-dimethylformamide and cooled to 0° C. Sodium hydride (1.2 eq; 60% suspended with mineral oil) is added followed by an alkyl chloride (1.05 eq) from Table 1 (CE), Table 2 (CC) or Table 3 (CT). After 2 hrs the reaction is warmed to room temperature, and then poured into ethyl acetate and water. After vigorous mixing the layers are allowed to settle, separated, and the aqueous layer is extracted again with ethyl acetate. Organic layers are pooled, dried over sodium sulfate, filtered to remove solids, and evaporated under reduced pressure to dryness to yield (1b) (See Scheme 1).

Nitrate Ester Formation (see also Kornblum, N, et. al., J. Am. Chem. Soc. 1966, 88, 1707-1711): (1b) (1.0 eq) is dissolved in acetonitrile (ACN) and silver nitrate (AgNO₃) is added (1.1 or 2.2 eq depending on the starting halide). The reaction is heated at 65° C. for 24 hrs, cooled, and silver halide precipitate is removed by filtration. The filtrate is evaporated to afford a residue that is purified by silica gel chromatography using 10% methanol in methylene chloride (v/v). Fractions containing pure product are pooled and evaporated under reduced pressure affording (1c). Table 4 lists starting materials (i.e., CE, CC, or CT) used in the synthesis of nitrooxy-alkylene-linked-coxib species-compounds 1-16.

TABLE 4
Starting Materials and Example Species-Compounds

| Starting Material | Ex. | Nitrooxy-Alkylene-Linked-Coxib Species-Compounds |
|---|---|---|
| CE1 | 1 | (N-((4-(5-(p-tolyl)-3-(trifluoromethyl)-1H-pyrazol-1- |
| | | yl)phenyl)sulfonyl)acetamido)methyl 2-(nitrooxy)acetate |
| CE2 | 2 | 1-(N-((4-(5-(p-tolyl)-3-(trifluoromethyl)-1H-pyrazol-1- |
| | | yl)phenyl)sulfonyl)acetamido)ethyl 2-(nitrooxy)acetate |
| CE3 | 3 | (N-((4-(5-(p-tolyl)-3-(trifluoromethyl)-1H-pyrazol-1- |
| | | yl)phenyl)sulfonyl)acetamido)methyl 2-(nitrooxy)propanoate |
| CE4 | 4 | (N-((4-(5-(p-tolyl)-3-(trifluoromethyl)-1H-pyrazol-1- |
| | | yl)phenyl)sulfonyl)acetamido)methyl 3-(nitrooxy)propanoate |
| CE5 | 5 | (N-((4-(5-(p-tolyl)-3-(trifluoromethyl)-1H-pyrazol-1- |
| | | yl)phenyl)sulfonyl)acetamido)methyl 4-(nitrooxy)butanoate |
| CE6 | 6 | (N-((4-(5-(p-tolyl)-3-(trifluoromethyl)-1H-pyrazol-1- |
| | | yl)phenyl)sulfonyl)acetamido)methyl 5-(nitrooxy)pentanoate |
| CE7 | 7 | (N-((4-(5-(p-tolyl)-3-(trifluoromethyl)-1H-pyrazol-1- |
| | | yl)phenyl)sulfonyl)acetamido)methyl 2-(nitrooxy)-2-phenylacetate |
| CC1 | 8 | 2-(nitrooxy)ethyl ((N-((4-(5-(p-tolyl)-3-(trifluoromethyl)-1H- |
| | | pyrazol-1-yl)phenyl)sulfonyl)acetamido)methyl) carbonate |
| CC2 | 9 | 1-(nitrooxy)propan-2-yl ((N-((4-(5-(p-tolyl)-3-(trifluoromethyl)- |
| | | 1H-pyrazol-1-yl)phenyl)sulfonyl)acetamido)methyl) carbonate |
| CC3 | 10 | 2-(nitrooxy)propyl ((N-((4-(5-(p-tolyl)-3-(trifluoromethyl)-1H- |
| | | pyrazol-1-yl)phenyl)sulfonyl)acetamido)methyl) carbonate |
| CC4 | 11 | 3-(nitrooxy)propyl ((N-((4-(5-(p-tolyl)-3-(trifluoromethyl)-1H- |
| | | pyrazol-1-yl)phenyl)sulfonyl)acetamido)methyl) carbonate |
| CC5 | 12 | 2,3-bis(nitrooxy)propyl ((N-((4-(5-(p-tolyl)-3-(trifluoromethyl)-1H- |
| | | pyrazol-1-yl)phenyl)sulfonyl)acetamido)methyl) carbonate |
| CC6 | 13 | 1,3-bis(nitrooxy)propan-2-yl ((N-(4-(5-(p-tolyl)-3- |
| | | (trifluoromethyl)-1H-pyrazol-1- |
| | | yl)phenyl)sulfonyl)acetamido)methyl) carbonate |
| CC7 | 14 | 4-(nitrooxy)butyl ((N-(4-(5-(p-tolyl)-3-(trifluoromethyl)-1H- |
| | | pyrazol-1-yl)phenyl)sulfonyl)acetamido)methyl) carbonate |
| CT1 | 15 | 2-((N-((4-(5-(p-tolyl)-3-(trifluoromethyl)-1H-pyrazol-1- |
| | | yl)phenyl)sulfonyl)acetamido)methoxy)ethyl nitrate |
| CT1 | 16 | 2-((N-((4-(5-(p-tolyl)-3-(trifluoromethyl)-1H-pyrazol-1- |
| | | yl)phenyl)sulfonyl)acetamido)methoxy)butyl nitrate |

### D. Definitions

The terms “substituent”, “radical”, “group”, “moiety” and “fragment” may be used interchangeably.

The term “hydrido” denotes a single —H atom (H) and may be used interchangeably with the symbol “H”. Hydrido may be attached, for example, to an oxygen atom to form a “hydroxy” radical (i.e., —OH), or two hydrido radicals may be attached to a carbon atom to form a “methylene” (—CH₂—) radical.

The terms “hydroxyl” and “hydroxy” may be used interchangeably.

If a substituent is described as being “optionally substituted,” the substituent may be either (1) not substituted or (2) substituted on a substitutable position. If a substitutable position is not substituted, the default substituent is H.

Singular forms “a” and “an” may include plural reference unless the context clearly dictates otherwise.

The number of carbon atoms in a substituent can be indicated by the prefix “C_{A-B}” where A is the minimum and B is the maximum number of carbon atoms in the substituent.

The term “halo” refers to fluoro (—F), chloro (—Cl), bromo (—Br), or iodo (—I).

The term “alkyl” denotes a linear or branched acyclic alkyl radical containing from 1 to about 15 carbon atoms. In some embodiments, alkyl is a C₁₋₁₀alkyl, C₁-alkyl, C₁₋₆alkyl or C₁₋₅alkyl radical. Examples of alkyl include, but are not limited to, methyl, ethyl, propyl, isopropyl, butyl, isobutyl, tert-butyl, sec-butyl, pentan-3-yl (i.e.,

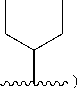 [SMILES: *C(CC)CC]

and the like.

The term “alkylcarbonyl” denotes an alkyl radical attached to carbonyl.

The term “hydroxyalkyl” embraces a radical wherein any one or more of an alkyl carbon is substituted with a hydroxyl radical as defined above, for example, monohydroxyalkyl, dihydroxyalkyl and trihydroxyalkyl. More specific examples of hydroxyalkyl include hydroxymethyl, hydroxyethyl and hydroxypropyl.

Hydroxyalkyl may be substituted with, for example, alkyl, hydroxyalkyl, hydroxyalkoxy, hydroxyalkoxyalkyl, amino, aminoalkyl, aryl, aralkyl and heterocyclyl. Further non-limiting examples include hydroxyalkyl substituted with methyl, isobutyl, benzyl, isopropyl, benzyl and sec-butyl.

The term “hydroxyalkoxy” denotes a hydroxy radical attached to an alkoxy radical.

The term “hydroxyalkoxyalkyl” denotes a hydroxyalkoxy radical attached to an alkyl radical. Non-limiting examples include hydroxyethyl-O-ethyl and hydroxylmethyl-O-ethyl.

Hydroxyalkoxyalkyl may, for example, be substituted with alkyl, hydroxyalkyl, hydroxyalkoxy, hydroxyalkoxyalkyl, amino, aminoalkyl, aryl, aralkyl and heterocyclyl. Further non-limiting examples include hydroxyalkoxyalkyl substituted with methyl, isobutyl, benzyl, isopropyl and sec-butyl. More specific non-limiting examples of substituted hydroxyalkoxyalkyl include hydroxyethyl-O-ethyl substituted with methyl, isobutyl, benzyl, isopropyl and sec-butyl.

The term “haloalkyl” embraces an alkyl radical wherein any one or more of the alkyl carbon atoms is substituted with halo as defined above. For example, monohaloalkyl, dihaloalkyl and trihaloalkyl. A monohaloalkyl radical, for one example, may have either a bromo, chloro or a fluoro atom within the radical. A dihalo radical may have two of the same halo radicals or a combination of different halo radicals. A trihaloalkyl radical may have three of the same halo radicals or a combination of different halo radicals. Non-limiting examples of haloalkyl include fluoromethyl, difluoromethyl, trifluoromethyl, chloromethyl, dichloromethyl, trichloromethyl, trifluoroethyl, pentafluoroethyl, heptafluoropropyl, difluorochloromethyl, dichlorofluoromethyl, difluoroethyl, difluoropropyl, dichloroethyl, dichloropropyl, iodomethyl, diiodomethyl and triiodomethyl.

The term “alkylene” denotes a divalent linear or branched saturated carbon chain containing from 2 to about 15 carbon atoms. The terms “alkylene” and “alkylenyl” may be used interchangeably. Non-limiting examples of alkylene radicals include methylene, ethylenyl

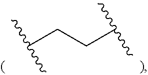 [SMILES: *CC*]

propylenyl,

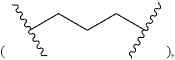 [SMILES: *CCC*]

butylenyl

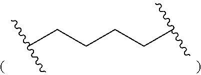 [SMILES: *CCCC*]

and pentylenyl

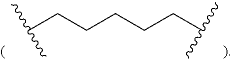 [SMILES: *CCCCC*]

One or more substitutable carbons in an alkylene radical may be replaced with, for example,—CH(Z⁴)—, —CH(Z⁴)—O—, —C(R⁴)₂—,

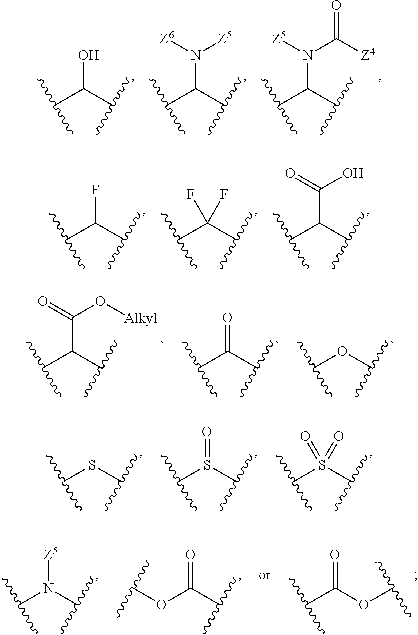

where Z⁴ may be, for example, H, alkyl, aryl, aralkyl, heterocyclyl (e.g., heteroaryl, more specifically phthalimido), alkyl-O—, alkyl-S—, alkyl-NH—, nitrooxy and nitrooxyalkylene, or Z⁴ may be taken together with Z⁵ or Z⁶ to form a cyclic ring (e.g, heterocyclyl); Z⁵ may be, for example, H, alkyl, hydroxyalkyl, aryl, heterocyclyl, alkylcarbonyl, arylcarbonyl, heterocyclylcarbonyl, carboxyalkylcarbonyl, alkyloxycarbonylalkylcarbonyl, alkylsulfonyl, arylsulfonyl and heteroarylsulfonyl, or Z⁵ may be taken together with Z⁴ or ZR⁶ to form a cyclic ring (e.g., heterocyclyl); and Z⁶ may be, for example, H, alkyl, hydroxyalkyl, aryl, heterocyclyl, alkylcarbonyl, arylcarbonyl, heterocyclylcarbonyl, carboxyalkylcarbonyl, alkyloxycarbonylalkylcarbonyl, alkylsulfonyl, arylsulfonyl and heteroarylsulfonyl, or R⁶ may be taken together with Z⁴ or Z⁵ to form a cyclic ring (e.g., heterocyclyl).

Examples of substituted alkylene include, ethyleneoxypropylene

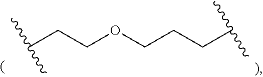 [SMILES: *CCCOCC*]

ethyleneoxycarbonylethylene

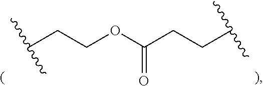 [SMILES: *CCOC(=O)CC*]

ethyleneoxy

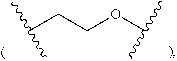 [SMILES: *CCO*]

ethyleneoxymethylene

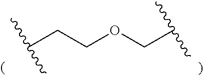 [SMILES: *CCOC*]

ethyleneoxypropylene

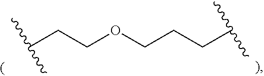 [SMILES: *CCCOCC*]

ethylenecarboyl

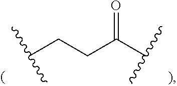 [SMILES: *CCC(*)=O]

ethylenethiocarbonyl

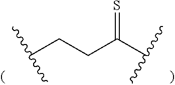 [SMILES: *CCC(*)=S]

and ethylenethionyl

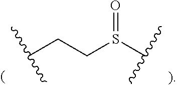 [SMILES: *CCS(*)=O]

One or more adjacent substitutable carbons in an alkylene radical may be replaced with a

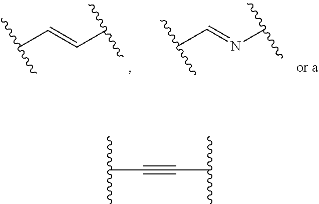

radical.

When one or more substitutable carbons in alkylene are substituted and the resulting radical has multiple orientations (e.g.,

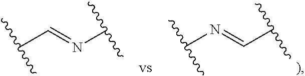

both orientations are embraced by the display of a single orientation.

If two adjacent carbons in an alkylene radical are replaced with heteroatoms, only suitable combinations are embraced. Non-limiting examples of suitable combinations, where two or more adjacent carbon atoms are replaced with O, N or S include:

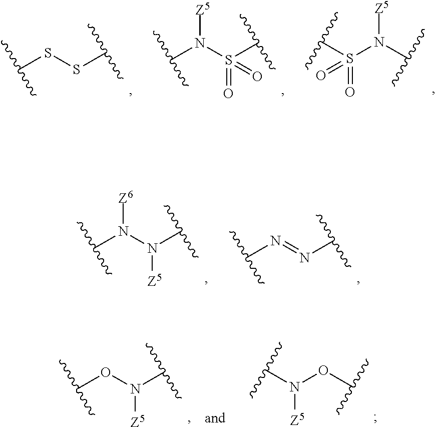

where Z⁵ and Z⁶ are defined as above.

Unsuitable combinations are synthetically unstable combinations and are not embraced by the current invention. Examples of unsuitable combinations include

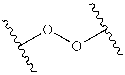 [SMILES: *OO*]

and geminal heteroatom combinations with

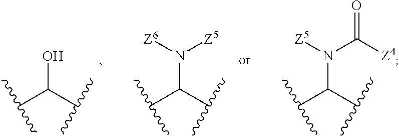

where Z⁴, Z⁵ and Z⁶ are defined as above.

The term “alkoxy” is RO— where R is alkyl as defined above. Non-limiting examples of alkoxy radicals include methoxy, ethoxy and propoxy. The terms “alkyloxy”, “alkoxy” and “alkyl-O—” may be used interchangeably.

The term “alkoxyalkyl” refers to an alkoxy moiety substituted with an alkyl radical. Examples of alkoxyalkyl radicals include methoxymethyl, methoxyethyl, methoxypropyl and ethoxyethyl.

The term “alkoxycarbonyl” refers to an alkoxy radical substituted with carbonyl. Non-limiting examples include methoxycarbonyl and ethoxycarbonyl.

The term “alkoxycarbonylalkyl” refers to an alkoxycarbonyl radical substituted with alkyl.

The term “alkyloxycarbonylalkylcarbonyl” refers to alkoxycarbonylalkyl radical substituted with carbonyl (e.g.,

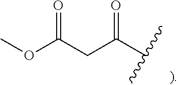 [SMILES: *C(=O)CC(=O)OC]

The term “alkenyl” refers to an unsaturated, acyclic hydrocarbon radical with at least one double bond. Such alkenyl radicals contain from 2 to about 15 carbon atoms.

The term “alkynyl” refers to an unsaturated, acyclic hydrocarbon radical with at least one triple bond. Such alkynyl radicals containing from 2 to about 15 carbon atoms. A non-limiting example is propargyl.

The term “cyano” denotes a carbon radical having three of four covalent bonds shared by a single nitrogen atom.

The term “carbonyl” denotes a carbon radical having two of four covalent bonds shared with a single oxygen atom.

The term “alkylcarbonyl” denotes an alkyl radical attached to a carbonyl radical.

The term “carbonylalkylene” denotes a carbonyl radical attached to an alkylene radical.

The term “carbonylalkylenecarbonyl” denotes a carbonylalkylene radical attached to a carbonyl radical.

The term “thiocarbonyl” denotes a carbon radical having two of four covalent bonds shared with a single sulfur atom.

The term “ureido” denotes

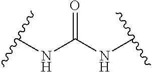 [SMILES: *NC(=O)N*]

and may be used interchangeably with carbamido.

The term “acyl”, is

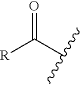

where R may be, for example, H, alkyl, nitrooxyalkylene, aryl and aralkyl. More specific examples of acyl include formyl, acetyl, benzoyl, nitrooxymethylcarbonyl and nitrooxyethylcarbonyl.

The term “acylamino” is

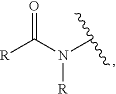

where R may be, for example, H, alkyl, nitrooxyalkylene, aryl and aralkyl. A more specific example of acylamino is acetylamino.

The term “carboxy” embraces a hydroxy radical attached to one of two unshared bonds in a carbonyl radical.

The term “carboxyalkylene” embraces a carboxy radical attached to an alkylene radical (e.g.,

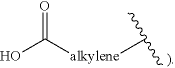

Non-limiting examples of carboxyalkylene include carboxymethylene and carboxyethylenyl. The terms “carboxyalkylene” and “hydroxycarbonylalkylene” may be used interchangeably.

The term “carboxyalkylenecarbonyl” denotes a carboxyalkylene radical attached to a carbonyl radical.

The term “thiocarboxy” embraces a hydroxyl radical, as defined above, attached to one of two unshared bonds in a thiocarbonyl radical.

The term “thiocarboxyalkyenel” embraces a thiocarboxy radical, as defined above, attached to an alkylene radical. Non-limiting examples include thiocarboxymethylene and thiocarboxyethylenyl.

The term “amido” embraces an amino radical attached to a parent molecular scaffold through carbonyl (e.g.,

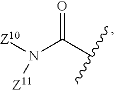

where Z¹⁰ and Z¹¹ may be, H, alkyl or aralkyl or Z¹⁰ may be taken together with Z¹¹ to form heterocyclyl, wherein at least one heteroatom is an amido nitrogen). The terms “amido” and “carboxamido” may be used interchangeably. Examples of amido radicals include monoalkylaminocarbonyl, dialkylaminocarbonyl. More specific examples of amido radicals include N-methylamino carbonyl and N,N-dimethylaminocarbonyl.

The term “carbamic” is

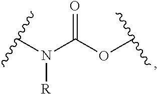

where R may be, for example, H, alkyl or acyl.

The term “cyclic ring” embraces any aromatic or non-aromatic cyclized carbon radical (e.g., aryl and cycloalkyl respectively) which may contain one or more ring heteroatoms (e.g., heteroaryl and heterocyclyl).

The term “cycloalkyl” embraces any monocyclic, bicyclic or tricyclic cyclized carbon radical of 3 to about 15 carbon atoms that is fully or partially saturated. Cycloalkyl may be attached to an aryl, cycloalkyl or a heterocyclyl radical in a fused or pendant manner.

Cycloalkyl may be substituted with alkyl, alkoxy, carboxyalkylene, hydroxyalkyl, amino, acylamino, amido, alkylamino, nitrooxyalkylene, nitrooxy, carbonyl, acyl, aralkyl, aryl, heterocyclyl or cycloalkyl.

The term “aryl” refers to any monocyclic, bicyclic or tricyclic cyclized carbon radical, wherein at least one ring is aromatic. An aromatic radical may be attached to a non-aromatic cycloalkyl or heterocyclyl radical in a fused or pendant manner. Examples of aryl radicals include, but are not limited to, phenyl and naphthyl.

The term “arylcarbonyl” denotes an aryl radical attached to a carbonyl radical. The terms “aroyl” and “arylcarbonyl” may be used interchangeably. Examples of arylcarbonyl include benzoyl and toluoyl.

The term “aralkyl” embraces aryl attached to an alkyl radical and may be used interchangeably with arylalkyl. Examples of aralkyl include benzyl, diphenylmethyl, triphenylmethyl, phenylethyl and diphenylethyl. The terms “benzyl” and “phenylmethyl” may be used interchangeably.

The term “heterocyclyl” refers to any monocyclic, bicyclic or tricyclic ring system having from 5 to about 15 ring members selected from carbon, nitrogen, sulfur and oxygen, wherein at least one ring member is a heteroatom. Heterocyclyl embraces a fully saturated, partially saturated and fully unsaturated radical (e.g., heteroaryl). Heterocyclyl may be fused or attached in a pendant manner to another heterocyclyl, aryl or cycloalkyl radical.

Heterocyclyl embraces combinations of different heteroatoms within the same cyclized ring system. When nitrogen is a ring member, heterocyclyl may be attached to the parent molecular scaffold through a ring nitrogen. Non-limiting examples of fully saturated five and six-membered heterocyclyl include: pyrrolidinyl, imidazolidinyl, piperidinyl, piperazinyl, tetrahydrofuranyl, morpholinyl and thiazolidinyl. Examples of partially saturated heterocyclyl include dihydrothiophenyl

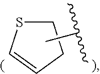

dihydropyranyl, dihydrofuranyl and dihydrothiazolyl.

Heterocyclyl may be substituted, for example, with alkyl, alkoxy, carboxyalkylene, hydroxyalkyl, amino, acylamino, amido, alkylamino, nitrooxyalkylene, nitrooxy, carbonyl, acyl, aralkyl, aryl, heterocyclyl or cycloalkyl. Non-limiting examples include, five-membered heterocyclyl substituted with hydroxyalkyl, alkoxyalkyl, acyl, carbonyl or alkylaminocarbonyl. More specifically, pyrrolidinyl may be substituted with hydroxyalkyl, alkoxyalkyl, acyl, carbonyl or alkylaminocarbonyl. Substituted and un-substituted 5-membered heterocyclyl may be fused or attached in a pendant manner to an additional heterocyclyl, aryl or cycloalkyl radical. For example, pyrrolidinyl-2,5-dione may be fused to phenyl giving isoindolinyl,1,3-dione (also termed “phthalimido”).

Six-membered heterocyclyl may be substituted with, for example, hydroxyalkyl, alkoxyalkyl, acyl, carbonyl or alkylaminocarbonyl. More specifically, piperidinyl, piperazinyl and morpholinyl may be substituted with hydroxyalkyl, alkoxyalkyl, acyl, carbonyl or alkylaminocarbonyl. Substituted and un-substituted 6-membered heterocyclyl may be fused or attached in a pendant manner to an additional heterocyclyl, aryl or cycloalkyl radical.

The term “heteroaryl” refers to an aromatic heterocyclyl radical. Heteroaryl may be fused or attached in a pendant manner to another heterocyclyl, aryl or cycloalkyl radical. Heteroaryl embraces combinations of different heteroatoms within the same cyclized radical. When nitrogen is a ring member, heteroaryl may be attached to the parent molecular scaffold through a ring nitrogen. Non-limiting examples of heteroaryl include pyridyl, thienyl, furanyl, pyrimidyl, imidazolyl, pyrazolyl, thiazolyl, thiadiazolyl, isothiazolyl, oxazolyl, isoxazoyl, pyrrolyl, pyridazinyl, pyrazinyl, quinolinyl, isoquinolinyl, benzofuranyl, benzothienyl, indolyl, benzothiazolyl, benzooxazolyl, benzimidazolyl, isoindolyl, benzotriazolyl

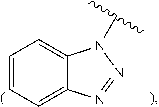 [SMILES: *n1nnc2ccccc21]

purinyl and thianaphthenyl. The term “heteroaryl” is also understood to include the N-oxide derivative of any nitrogen containing heteroaryl.

The term “alkylamino” embraces an alkyl radical attached to a molecular scaffold through an amino radical (e.g., alkyl-NH-scaffold). Specific non-limiting examples of alkylamino include N,N-dimethylamino-scaffold and N-methylamino-scaffold.

The term “aminoalkyl” embraces an amino radical attached to a molecular scaffold through an alkyl radical (e.g., NH₂-alkyl-scaffold).

The term “aralkoxy” embraces an arylalkyl radical attached through an oxygen atom to the parent molecular scaffold. The terms “arylalkoxy” and “aralkoxy” may be used interchangeably.

The term “aryloxy” is RO—, where R is aryl.

The term “arylthio” is RS—, where R is aryl.

The term “alkylthio” is RS—, where R is alkyl (e.g., alkyl-S-scaffold).

The term “thiolalkyl” is HSR—, where R is alkyl (e.g., HS-alkyl-scaffold).

The term “aryloxyalkyl” embraces an aryloxy radical attached to an alkyl radical.

The term “sulfonyl” is —SO₂—.

The term “alkylsulfonyl” embraces an alkyl radical attached to a sulfonyl radical, where alkyl is defined as above.

The term “arylsulfonyl” embraces an aryl radical attached to a sulfonyl radical.

The term “heteroarylsulfonyl” embraces a heteroaryl radical attached to a sulfonyl radical.

The term “alkylsulfonylalkyl”, embraces an alkylsulfonyl radical attached to an alkyl radical, where alkyl is defined as above.

The term “haloalkylsulfonyl” embraces a haloalkyl radical attached to a sulfonyl radical, where haloalkyl is defined as above.

The term “nitrooxy” denotes

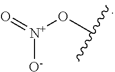 [SMILES: *O[N+](=O)[O-]]

The term “nitrooxyalkylene” embraces a nitrooxy radical attached to an alkylene radical (e.g.,

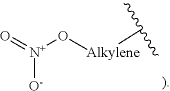

The term “sulfonamide” denotes sulfonyl attached to an amino radical. For example: NH₂SO₂— and —NHSO₂—. Sulfonamide may be used interchangeably with sulfamyl, sulfonamido and aminosulfonyl.

The term “nitrooxy-alkylene-linked-coxib” refers to a nitrooxy-containing radical that is attached to sulfonamide nitrogen of celecoxib through an alkylene radical. For example, when a nitrooxy radical is attached to celecoxib through a methylene radical, connectivity is termed “nitrooxy-methylene-linked-coxib”:

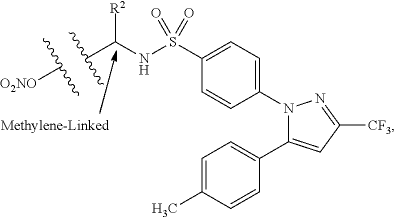

where R² may be, for example, H, alkyl, aryl, heterocyclyl or nitrooxyalkylene.

The term “imine” denotes a compound containing the structure >C═N—.

The term “coxib” is any member of a class of nonsteroidal anti-inflammatory drugs that causes fewer gastrointestinal side effects by selective inhibition of prostaglandin formation. The terms “coxib” and “selective COX-2 inhibitor” may be used interchangeably.

The term “pharmaceutically-acceptable” means suitable for use in pharmaceutical preparations, generally considered as safe for such use, officially approved by a regulatory agency of a national or state government for such use, or being listed in the U.S. Pharmacopoeia or other generally recognized pharmacopoeia for use in animals and more particularly in humans.

The term “pharmaceutically-acceptable salt” refers to a salt which may enhance desired pharmacological activity or may enhance stability of a compound. Examples of pharmaceutically-acceptable salts include acid addition salts formed with inorganic or organic acids, metal salts, and amine salts. Examples of acid addition salts formed with inorganic acids include salts with hydrochloric acid, hydrobromic acid, hydriodic acid, sulfuric acid, nitric acid, and phosphoric acid. Examples of acid addition salts formed with organic acids include acetic acid, propionic acid, hexanoic acid, heptanoic acid, cyclopentanepropionic acid, glycolic acid, pyruvic acid, lactic acid, citric acid, malonic acid, succinic acid, malic acid, maleic acid, fumaric acid, tartaric acid, citric acid, benzoic acid, o-(4-hydroxy-benzoyl)-benzoic acid, cinnamic acid, mandelic acid, methanesulfonic acid, ethanesulfonic acid, 1,2-ethanedisulfonic acid, 2-hydroxyethane-sulfonic acid, benzenesulfonic acid, p-chlorobenzenesulfonic acid, 2-naphthalenesulfonic acid, p-toluenesulfonic acid, camphorsulfonic acid, 4-methyl-bicyclo[2.2.2]oct-2-ene1-carboxylic acid, glucoheptonic acid, 4,4′ -methylenebis(3-hydroxy-2-naphthoic) acid, 3-phenylpropionic acid, trimethylacetic acid, tertiary butylacetic acid, lauryl sulfuric acid, gluconic acid, glutamic acid, hydroxynaphthoic acids, salicylic acid, stearic acid, and muconic acid. Examples of metal salts include salts with sodium, potassium, calcium, magnesium, aluminum, iron, barium, bismuth, lithium, and zinc ions. Examples of amine salts include salts with ammonia, arecoline, arginine, benethamine, benzathamine, betaine, chloroprocaine, choline, clemizole, cytosine, deanol, diethanolamine, diethylamine, diethylamine, diethylaminoethanol, epolamine, ethanolamine, ethylenediamine, guanine, imidazole, lysine, meglumine, morpholineethanol, niacinamide, piperazine, procaine, pyridoxine, tert-butlamine (erbumine), thiamine, thymine, trolamine, tromethamine, and uracil.

The term “therapeutically-effective amount” refers to an amount of a compound that, when administered to a subject for treating a disease, is sufficient to effect treatment for the disease. “Therapeutically effective amount” can vary depending on the compound, the disease and its severity, the age, the weight, etc. of the subject to be treated.

A compound of the present invention can exist in tautomeric, geometric or stereoisomeric forms. An ester, metabolite, oxime, prodrug, onium, hydrate, solvate and N-oxide of a compound of Formula I are also embraced by the invention. The present invention contemplates all such compounds, including cis- and trans-geometric isomers, R- and S-enantiomers, diastereomers, d-isomers, l-isomers, mixtures of isomers and racemates thereof, as falling within the scope of the invention.

The term “solvate” denotes a molecular or ionic complex of molecules or ions of solvent with those of a compound of the present invention. The term “solvate” embraces the term “hydrate”.

The term “hydrate” denotes a compound of the present invention containing water combined in the molecular form.

Some of the compounds described contain one or more stereocenters and are meant to include R, S and mixtures of R and S forms for each stereocenter present.

List of Suitable Protecting Groups and Abbreviations:
Acetyl (Ac)
Acylals
Benzoyl (Bz)
Benzyl (Bn, Bnl)
Benzyl esters
Carbamate
Carbobenzyloxy (Cbz)
Dimethoxytrityl, [bis-(4-methoxyphenyl)phenylmethyl] (DMT)
Dithianes
Ethoxyethyl ethers (EE)
Methoxymethyl ether (MOM)
Methoxytrityl [(4-methoxyphenyl)diphenylmethyl], (MMT)
Methyl Ethers
Methyl (Me)
Methyl esters
Methylthiomethyl ether
Orthoesters
Oxazoline
Pivaloyl (Piv)
Phthalimido
p-Methoxybenzyl carbonyl (Moz or MeOZ)
p-Methoxybenzyl (PMB)
Propargyl alcohols
Silyl groups (e.g., trimethylsilyl (TMS), tert-butyldimethylsilyl (TBDMS), tri-iso-propylsilyloxymethyl (TOM) and triisopropylsilyl (TIPS))
Silyl esters
tert-Butyl esters
tert-Butyloxycarbonyl (BOC or tBOC)
Tetrahydropyranyl (THP)
Tosyl (Ts or Tos)
Trimethylsilylethoxymethyl (SEM)
Trityl (triphenylmethyl, Tr)
β-Methoxyethoxymethyl ether (MEM)
(4-nitrophenyl)sulfonyl or (4-nitrophenyl)(dioxido)-lambda(6)-sulfanyl) (Nosyl)
2-cyanoethyl
2-nitrophenylsulfenyl (Nps)
3,4-Dimethoxybenzyl (DMPM)
9-Fluorenylmethyloxycarbonyl (FMOC)

List of abbreviations:
ACN acetonitrile
DCC dicyclohexylcarbodiimide
DCI dicyclohexylcarbodiimide
DCM dichloromethane or methylenechloride
DIPEA diisopropylethylamine
DMAP 4-dimethylaminopyridine or N,N-dimethylaminopyridine
DME 1,2-dimethoxyethane
DMF N,N-dimethylformamide
DMSO dimethylsulfoxide
eq. equivalents
EtOAC ethyl acetate
EtOH ethanol
Fmoc fluorenylmethyloxycarbonyl chloride
HPLC high performance liquid chromatography
hr hour
hrs hours
K₂CO₃ potassium carbonate
LC/MS liquid chromatography mass spectrometry
LC/MS/MS liquid chromatography tandem mass spectrometry
MeOH methanol
MgSO₄ magnesium sulfate
min. minute(s)
mL milliliter
mmol millimole
Na₂S₂O₃ sodium thiosulfate
Na₂SO₄ sodium sulfate
NaH sodium hydride
NaHCO₃ sodium bicarbonate
NaI sodium Iodide
NaIO₄ sodium periodate
NaOCH₃ sodium methoxide
NBS N-bromosuccinimide
NIS N-iodosuccinimide
NMR nuclear magnetic resonance
NO nitric oxide
psi pounds per square inch
PyBOP benzotriazole-1-yl-oxy-tris-(dimethylamino)-phosphonium hexafluorophosphate
RuCl₃ ruthenium trichloride hydrate
TEA triethylamine
TFA trifluoroacetic acid
THF tetrahydrofuran
TLC thin layer chromatography
TSA p-toluenesulfonic acid

### E. Method of Treatment

A compound of the structural formulae herein is meant to include a pharmaceutically acceptable salt, or solvate of a compound or salt, of the structural formulae herein.

The present invention further provides methods for treating a disease condition in a subject having or susceptible to having such a disease condition, by administering to the subject a therapeutically-effective amount of one or more compounds as described in the structural formulae herein. In one embodiment, the treatment is preventative treatment. In another embodiment, the treatment is palliative treatment. In another embodiment, the treatment is restorative treatment. In another embodiment, the subject is a mammal. In yet another embodiment, the subject is a human.

1. Conditions

The conditions that can be treated in accordance with the present invention include, but are not limited to, autoimmune disorders, chronic inflammatory disorders, acute inflammatory disorders, auto-inflammatory disorders, pain, cancer, neoplasia, lung cancer, colorectal cancer, and the like.

In some embodiments, methods described herein are used to treat or prevent a disease condition comprising administering to a subject in need thereof a therapeutically effective amount of a compound of the structural formulae herein, wherein the condition is selected from the group consisting of non-small cell lung cancer, skin cancer, liver cancer, metastatic colorectal cancer (and FAP), renal cell cancer, glioblastoma, squamous cell cancer, bladder cancer, breast cancer, biliary tract cancer, cervical cancer, prostate cancer, small cell lung cancer, ovarian cancer, pancreatic cancer, gastrointestinal cancer, and CNS cancer.

In some embodiments, methods described herein are used to treat a disease condition comprising administering to a subject in need thereof a therapeutically effective amount of a compound of the structural formulae herein, wherein the condition is selected from the group consisting of actinic keratosis, cystic fibrosis, and acne.

In some embodiments, methods described herein are used for healing wounds by administering to a subject in need thereof a therapeutically effective amount of a compound of the structural formulae herein.

In some embodiments, methods described herein are used to treat a disease condition comprising administering to a subject in need thereof a therapeutically effective amount of a compound of the structural formulae herein, wherein the condition is non-small cell lung cancer.

In some embodiments the methods described herein are used to treat patients with disorders arising from dysregulated enzymes, and/or inflammatory mediator production, stability, secretion, and posttranslational processing. Examples of inflammatory mediators that may be dysregulated include nitric oxide, prostaglandins, and leukotrienes. Examples of enzymes include cyclo-oxygenase and nitric oxide synthase.

In some embodiments, the methods described herein are used to treat a patient in need thereof suffering from an autoimmune disorder, chronic, and/or acute inflammatory disorder, and/or auto-inflammatory disorder. Examples of disorders include, but are not limited to arthritis, rheumatoid arthritis, osteoarthritis, juvenile arthritis, psoriatic arthritis.

In some embodiments, the methods described herein can be used to treat a patient in need thereof, and suffering from neoplasia. Examples of these conditions include but are not limited to the following:

acral lentiginous melanoma
actinic keratoses
adenocarcinoma
adenoid cycstic carcinoma
adenomas
adenosarcoma
adenosquamous carcinoma
astrocytic tumors
bartholin gland carcinoma
basal cell carcinoma
bladder cancer
breast cancer
biliary tract cancer
bronchial gland carcinomas
capillary
carcinoids
carcinoma
carcinosarcoma
cavernous
cervical cancer
cholangiocarcinoma
chondosarcoma
choroid plexus papilloma/carcinoma
clear cell carcinoma
CNS cancer
cystadenoma
endodermal sinus tumor
endometrial hyperplasia
endometrial stromal sarcoma
endometrioid adenocarcinoma
ependymal
epitheloid
Ewing's sarcoma
familial adenomatous polyposis (FAP)
fibrolamellar carcinoma
focal nodular hyperplasia
gastrinoma
gastrointestinal cancer
germ cell tumors
glioblastoma
glucagonoma
hemangiblastomas
hemangioendothelioma
hemangiomas
hepatic adenoma
hepatic adenomatosis
hepatocellular carcinoma
insulinoma
intaepithelial neoplasia
interepithelial squamous cell neoplasia
invasive squamous cell carcinoma
large cell carcinoma
leiomyosarcoma
lentigo maligna melanomas
liver cancer
malignant melanoma
malignant mesothelial tumors
medulloblastoma
medulloepithelioma
melanoma
meningeal
mesothelial
metastatic carcinoma
metastatic colorectal cancer
mucoepidermoid carcinoma
neuroblastoma
neuroepithelial adenocarcinoma nodular melanoma
non-small cell lung cancer
oat cell carcinoma
oligodendroglial
osteosarcoma
ovarian cancer
pancreatic cancer
papillary serous adenocarcinoma
pineal cell
pituitary tumors
plasmacytoma
prostate cancer
pseudosarcoma
pulmonary blastoma
renal cell carcinoma
retinoblastoma
rhabdomyosarcoma
sarcoma
serous carcinoma
skin cancer
small cell carcinoma
small cell lung cancer
soft tissue carcinomas
somatostatin-secreting tumor
squamous carcinoma
squamous cell carcinoma
submesothelial
superficial spreading melanoma
undifferentiatied carcinoma
uveal melanoma
verrucous carcinoma
vipoma
well differentiated carcinoma
Wilm's tumor

The term patient refers to both humans and non-human animals with the abovementioned conditions. Non-human animals could be companion animals such as, but not limited to, canine and feline species.

2. Subjects

Suitable subjects to be treated according to the present invention include mammalian subjects. Mammals according to the present invention include, but are not limited to, human, canine, feline, bovine, caprine, equine, ovine, porcine, rodents, lagomorphs, primates, and the like, and encompass mammals in utero. Subjects may be of either gender and at any stage of development.

3. Administration and Dosing

A compound of the present invention may be administered in the form of a prodrug in a therapeutically effective amount.

The compounds of the present invention can be administered by any suitable route in the form of a pharmaceutical composition adapted to such a route and in a dose effective for the treatment intended. Therapeutically effective doses of the compounds of the present invention required to prevent or arrest the progress of, or to treat the medical condition, are readily ascertained by one of ordinary skill in the art using preclinical and clinical approaches familiar to the medicinal arts.

For convenience the compounds of the present invention can be administered in a unit dosage form. If desired, multiple doses per day of the unit dosage form can be used to increase the total daily dose. The unit dosage form, for example, may be a tablet or capsule containing about 0.01, 0.05, 0.1, 0.5, 1, 5, 10, 15, 20, 25, 30, 35, 40, 45, 50, 55, 60, 65, 70, 75, 80, 85, 90, 95, 100, 125, 150, 175, 200, 250, or 500 mg of the compound of the present invention. In one embodiment, the unit dosage form contains from about 0.01 mg to about 500 mg of the compound of the present invention. In another embodiment, the unit dosage form contains from about 0.02 to about 400 mg of the compound of the present invention. In another embodiment, the unit dosage form contains from about 0.05 mg to about 250 mg of the compound of the present invention. In another embodiment, the unit dosage form contains from about 0.1 mg to about 200 mg of the compound of the present invention. In another embodiment, the unit dosage form contains from about 0.5 mg to about 150 mg of the compound of the present invention. In another embodiment, the unit dosage form contains from about 1.0 mg to about 100 mg of the compound of the present invention.

The dosage regimen for the compounds and/or compositions containing the compounds is based on a variety of factors, including the type, age, weight, sex, and medical condition of the patient; the severity of the condition; the route of administration; and the activity of the particular compound employed. Thus the dosage regimen may vary based on the specific situation. Dosage levels from about 0.001 mg to about 100 mg of the compound of the present invention per kilogram of body weight per day are useful in the treatment of the above-indicated conditions. In one embodiment, the total daily dose of the compound of the present invention (administered in single or divided doses) is typically from about 0.001 mg/kg to about 20 mg/kg (i.e., mg compound/kg body weight). In another embodiment, the total daily dose of the compound of the present invention is from about 0.005 mg/kg to about 10 mg/kg. In another embodiment, the total daily dose is from about 0.005 mg/kg to about 5 mg/kg. In another embodiment, the total daily dose is from about 0.01 mg/kg to about 1 mg/kg. In another embodiment, the total daily dose is from about 0.8 mg/kg to about 15 mg/kg. In another embodiment, the total daily dose is from about 0.2 mg/kg to about 4 mg/kg. These dosages are based on an average human subject having a weight of about 65 kg to about 75 kg. A physician will readily be able to determine doses for subjects whose weight falls outside of this range, such as infants. The administration of the compound of the present invention can be repeated a plurality of times in a day (typically no greater than 4 times) to achieve the desired daily dose.

The present invention further comprises use of a compound of the present invention as a medicament (such as a unit dosage tablet or unit dosage capsule).

In another embodiment, the present invention comprises the use of a compound of the present invention for the manufacture of a medicament (such as a unit dosage tablet or unit dosage capsule) to treat one or more of the conditions previously identified in the above sections discussing methods of treatment. In one embodiment, the condition is cancer. In another embodiment the condition is an inflammatory condition.

### F. Pharmaceutical Compositions

For treatment of the conditions referred to above, the compounds described herein can be administered as follows:

1. Oral Administration

The compounds of the present invention may be administered orally, including by swallowing, so that the compound enters the gastrointestinal tract, or absorbed into the blood stream directly from the mouth (e.g., buccal or sublingual administration).

Suitable compositions for oral administration include solid formulations such as tablets, lozenges, pills, cachets, and hard and soft capsules, which can contain liquids, gels, or powders.

Compositions for oral administration may be formulated as immediate or modified release, including delayed or sustained release, optionally with enteric coating.

Liquid formulations can include solutions, syrups, and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

In a tablet dosage form the amount of drug present may be from about 0.05% to about 95% by weight, more typically from about 2% to about 50% by weight of the dosage form. In addition, tablets may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch, and the like.

Suitable lubricants, for use in a tablet, may be present in amounts from about 0.1% to about 5% by weight and include calcium, zinc, or magnesium stearate, sodium stearyl fumarate, and the like.

Suitable binders, for use in a tablet, include gelatin, polyethylene glycol, sugars, gums, starch, hydroxypropyl cellulose, and the like. Suitable diluents, for use in a tablet, include mannitol, xylitol, lactose, dextrose, sucrose, sorbitol, and starch.

Suitable surface active agents and glidants, for use in a tablet, may be present in amounts from about 0.1% to about 3% by weight and include polysorbate 80, sodium dodecyl sulfate, talc, and silicon dioxide.

In another embodiment, a pharmaceutical composition comprises a therapeutically effective amount of a compound of the structural formulae herein or a pharmaceutically acceptable salt thereof, and a pharmaceutically acceptable carrier.

2. Parenteral Administration

Compounds of the present invention may be administered directly into the blood stream, muscle, or internal organs. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods.

Compositions for parenteral administration may be formulated as immediate or modified release, including delayed or sustained release.

Most parenteral formulations are aqueous solutions containing excipients, including salts, buffering agents, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

3. Topical Administration

Compounds of the present invention may be administered topically to the skin or transdermally. Formulations for this topical administration can include lotions, solutions, creams, gels, hydrogels, ointments, foams, implants, patches, and the like. Pharmaceutically acceptable carriers for topical administration formulations can include water, alcohol, mineral oil, glycerin, polyethylene glycol, and the like. Topical administration can also be performed by electroporation, iontophoresis, phonophoresis, and the like.

Compositions for topical administration may be formulated as immediate or modified release, including delayed or sustained release.

4. Rectal Administration

Suppositories for rectal administration of the compounds of the present invention can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature and which will therefore melt in the rectum and release the drug.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art, and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

### G. Combinations and Combination Therapy

The compounds of the present invention can be used, alone or in combination with other pharmaceutically active compounds, to treat conditions such as those previously described above. The compound(s) of the present invention and other pharmaceutically active compound(s) can be administered simultaneously (either in the same dosage form or in separate dosage forms) or sequentially. Accordingly, in one embodiment, the present invention comprises methods for treating a condition by administering to the subject a therapeutically-effective amount of one or more compounds of the present invention, and one or more additional pharmaceutically active compounds.

In another embodiment, there is provided a pharmaceutical composition comprising one or more compounds of the present invention, one or more additional pharmaceutically active compounds, and a pharmaceutically acceptable carrier.

In another embodiment, the one or more additional pharmaceutically active compounds is selected from the group consisting of anti-inflammatory drugs, cytostatic drugs, cytotoxic drugs, anti-proliferative agents, and angiogenesis inhibitors.

In another embodiment, the one or more additional pharmaceutically active compounds is selected from the group consisting of anti-cancer drugs and anti-inflammatory drugs.

NO-releasing coxib conjugate described herein are also optionally used in combination with other therapeutic reagents that are selected for their therapeutic value for the condition to be treated. In general, the compounds described herein and, in embodiments where combinational therapy is employed, other agents do not have to be administered in the same pharmaceutical composition and, because of different physical and chemical characteristics, are optionally administered by different routes. The initial administration is generally made according to established protocols and then, based upon the observed effects, the dosage, modes of administration and times of administration subsequently modified. In certain instances, it is appropriate to administer an NO-releasing coxib conjugate, as described herein, in combination with another therapeutic agent or NO-releasing coxib conjugate. By way of example only, the therapeutic effectiveness of an NO-releasing coxib conjugate is enhanced by administration of another therapeutic agent (which also includes a therapeutic regimen) that also has therapeutic benefit. Regardless of the disease, disorder or condition being treated, the overall benefit experienced by the patient is either simply additive of the two therapeutic agents or the patient experiences an enhanced benefit.

Therapeutically effective dosages vary when the drugs are used in treatment combinations. Methods for experimentally determining therapeutically effective dosages of drugs and other agents for use in combination treatment regimens are documented methodologies. Combination treatment further includes periodic treatments that start and stop at various times to assist with the clinical management of the patient. In any case, the multiple therapeutic agents (one of which is an NO-releasing coxib conjugate as described herein) are administered in any order, or even simultaneously. If simultaneously, the multiple therapeutic agents are optionally provided in a single, unified form, or in multiple forms (by way of example only, either as a single pill or as two separate pills).

In some embodiments, one of the therapeutic agents is given in multiple doses, or both are given as multiple doses. If not simultaneous, the timing between the multiple doses optionally varies from more than zero weeks to less than twelve weeks.

In addition, the combination methods, compositions and formulations are not to be limited to the use of only two agents, the use of multiple therapeutic combinations are also envisioned. It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time period between the multiple administration steps ranges from a few minutes to several hours, depending upon the properties of each pharmaceutical agent, such as potency, solubility, bioavailability, plasma half-life, and kinetic profile of the pharmaceutical agent.

In another embodiment, an NO-releasing coxib conjugate is optionally used in combination with procedures that provide additional benefit to the patient. An NO-releasing coxib conjugate, and any additional therapies are optionally administered before, during or after the occurrence of a disease or condition, and the timing of administering the composition containing an NO-releasing coxib varies in some embodiments. Thus, for example, an NO-releasing coxib conjugate is used as a prophylactic, and is administered continuously to subjects with a propensity to develop conditions or diseases in order to prevent the occurrence of the disease or condition. An NO-releasing coxib conjugate is optionally administered to a subject during or as soon as possible after the onset of the symptoms. While embodiments of the present invention have been shown, and described herein, it will be obvious to those skilled in the art that such embodiments are provided by way of example only. Numerous variations, changes, and substitutions will now occur to those skilled in the art without departing from the invention. It should be understood that in some embodiments of the invention various alternatives to the embodiments described herein are employed in practicing the invention.

A NO-releasing coxib conjugate can be used in combination with anti-cancer drugs, including but not limited to the following classes: alkylating agents, anti-metabolites, plant alkaloids, and terpenoids, topoisomerase inhibitors, cytotoxic antibiotics, angiogenesis inhibitors, and tyrosine kinase inhibitors.

For use in cancer and neoplastic diseases, an NO-releasing coxib conjugate may be optimally used together with one or more of the following non-limiting examples of anti-cancer agents. As a first example, alkylating agents include but are not limited to cisplatin (PLATIN), carboplatin (PARAPLATIN), streptozocin (ZANOSAR), busulfan (MYLERAN), and cyclophosphamide (ENDOXAN). As a second example, anti-metabolites include but are not limited to mercaptopurine (PURINETHOL), thioguanine, pentostatin (NIPENT), cytosine arabinoside (ARA-C), and methotrexate (RHEUMATREX). As a third example, plant alkaloids and terpenoids include but are not limited to vincristine (ONCOVIN), vinblastine, and paclitaxel (TAXOL). As a fourth example, topoisomerase inhibitors include but are not limited to irinotecan (CAMPTOSAR), topotecan (HYCAMTIN), and etoposide (EPOSIN). As a fifth example, cytotoxic antibiotics include but are not limited to actinomycin D (COSMEGEN), doxorubicin (ADRIAMYCIN), bleomycin (BLENOXANE), and mitomycin (MITOSOL). As a sixth example, angiogenesis inhibitors include but are not limited to sunitinib (SUTENT) and bevacizumab (AVASTIN). As a seventh example, tyrosine kinase inhibitors include but are not limited to imatinib (GLEEVEC), erlotinib (TARCEVA), lapatininb (TYKERB), and axitinib (INLYTA). As an eighth example, EGFR inhibitors include but are not limited to the monoclonal antibody cetuximab (ERBITUX). As a nineth example, agents that target HER2 include but are not limited to the monoclonal antibodies pertuzumab (PERJETA) and trastuzumab (HERCEPTIN) which have strong co-expression links to COX-2 in prostrate and breast cancer.

Where a subject is suffering from or at risk of suffering from an inflammatory condition, an NO-releasing coxib conjugate described herein is optionally used together with one or more agents or methods for treating an inflammatory condition in any combination. Therapeutic agents/treatments for treating an autoimmune and/or inflammatory condition include, but are not limited to any of the following examples. As a first example, corticosteroids include but are not limited to cortisone, dexamethasone, and methylprednisolone. As a second example, nonsteroidal anti-inflammatory drugs (NSAID's) include but are not limited to ibuprofen, naproxen, acetaminophen, aspirin, fenoprofen (NALFON), flurbiprofen (ANSAID), ketoprofen, oxaprozin (DAYPRO), diclofenac sodium (VOLTAREN), diclofenac potassium (CATAFLAM), etodolac (LODINE), indomethacin (INDOCIN), ketorolac (TORADOL), sulindac (CLINORIL), tolmetin (TOLECTIN), meclofenamate (MECLOMEN), mefenamic acid (PONSTEL), nabumetone (RELAFEN), and piroxicam (FELDENE). As a third example, immunosuppressants include but are not limited to methotrexate (RHEUMATREX), leflunomide (ARAVA), azathioprine (IMURAN), cyclosporine (NEORAL, SANDIMMUNE), tacrolimus, and cyclophosphamide (CYTOXAN). As a fourth example, CD20 blockers include but are not limited to rituximab (RITUXAN). As a fifth example, Tumor Necrosis Factor (TNF) blockers include but are not limited to etanercept (ENBREL), infliximab (REMICADE), and adalimumab (HUMIRA). As a sixth example, interleukin-1 receptor antagonists include but are not limited to anakinra (KINERET). As a seventh example, interleukin-6 inhibitors include but are not limited to tocilizumab (ACTEMRA). As an eighth example, interleukin-17 inhibitors include but are not limited to AIN457. As a ninth example, Janus kinase inhibitors include but are not limited to tasocitinib. As a tenth example, syk inhibitors include but are not limited to fostamatinib.

### H. Kits

The present invention further comprises kits that are suitable for use in performing the methods of treatment or prevention described above. In one embodiment, the kit contains a first dosage form comprising one or more of the compounds of the present invention, and a container for the dosage, in quantities sufficient to carry out the methods of the present invention.

### I. Biological Assays

### Compound Metabolism in Plasma & Microsomal Stability

The present invention includes compounds that are enzymatically activated in vivo to produce celecoxib. Compounds are analyzed, after incubation in plasma, for the rate of disappearance of the compound species and appearance of celecoxib and/or intermediate compounds.

Compounds are dissolved in DMSO to make 30 mM stocks. Compounds (2 μL) are incubated with pure rat or human plasma (98 μL; 600 μM) at 37° C. for various time points (T=3 h, 6 h, and 24 h). Reactions are stopped with 1.0 N HCl (400 μL) and extracted with ethyl acetate (500 μL). Each tube is vortexed for three 5-second intervals and then the tubes are spun down at maximum speed (13,000 rpm) for 10 minutes to pellet protein and separate the layers. The ethyl acetate layer is separated (top 300 μL), concentrated, diluted with 30 μL methanol and analyzed using HPLC by UV (254 nm) for the presence of starting compound, celecoxib and intermediate compounds.

Compounds (1 μM) are incubated, in triplicate, in rat plasma or liver microsomes at 37° C., and analyzed by LC/MS/MS (T=0, 10, 20, 30, 45 and 60 min). Reaction is quenched by acetonitrile. Analysis used are standard reverse phase HPLC and API 4000 triple quadrupole mass spectrometry. Elimination rate constant, in vitro half-life, and intrinsic clearance are calculated from results.

### Rat Air-Pouch Model for Acute Inflammation

A study of celecoxib release is assessed by measurement of PGE2 levels, which are indicative of inflammatory response.

Animals: Sprague-Dawley rats (Charles River Laboratories., R #3234, PO #738990, male, 160-180 g) are received, individually examined and housed in cages of five rats each. The rats are ear notched for identification purposes.

Compounds and dosing solutions: The vehicle is prepared by dissolving 40 g (2-hydroxypropyl)-β-cyclodextrin (HP-β-CD, Sigma, Cat. 332593, lot MKBJ5858V) in 160 mL sterile saline for injection, USP (Hospira, lot 26-801-FW) making a 25% solution which is filter sterilized (0.2 μm, Nalgene, Cat. 151-4020, lot 1095610). A 1% carrageenan solution is prepared by dissolving 0.6 λ-carrageenan (Fluka, Cat. 22049, lot 1318338) in hot 60 mL sterile saline for injection, USP. This solution is stored at 4-8° C. Test compounds are dissolved in DMSO (Fisher Scientific, Cat. D128-500, lot 874999) to make 75 mM stocks. 0.25 mL of compound DMSO stocks are mixed with 12.5 mL of HP-β-CD solution at 50° C. (maximum DMSO concentration is 2% of the final volume of vehicle,). Final concentration of all test compounds is 1.5 mM and compounds are dosed within 2 h of preparation at 0.01 mmol/kg (12 nmol of test compound per rat).

Day 0—Air pouch initiation: The rats are anesthetized in a biological cabinet, the nape of the neck is cleansed with 70% isopropanol (Butler Schein Animal Health, Cat. 002498, lot 29EMS07104547) followed by 1% povidone-iodine solution (Ricca Chemical Co., Cat. 3955-16, lot 2205469). Twenty mL of sterile (0.22 μm, Millipore, Cat. SLGP033RS, lot R2KA55925, exp 08/2015) air is injected subcutaneously (SC) using a 23G x 11/2 inch needle fixed to a 20 mL syringe. The rats are returned to routine housing.

Day 3—Air pouch maintenance: The rats are anesthetized in a biological cabinet, the nape of the neck is cleansed with 70% isopropanol followed by 1% povidone-iodine solution. Ten mL of sterile air is injected SC using a 23 G×1½ inch needle fixed to a 20 mL syringe. The rats are returned to routine housing in clean cages.

Day 6—Compound administration and carrageenan insult: At commencement of the study, each rat is weighed and sorted into treatment groups of 5 rats/group based upon average weight. Each rat is dosed orally via gavage at 6.809 mL/kg (1.6 mL/235 g) with their respective test material/vehicle. Two hours after test material/vehicle administration, the rats are injected with 1.0 mL of the room temperature 1% carrageenan saline solution into the air pouch. Four hours after carrageenan injection, the rats are anesthetized, and 5 mL of the exudate buffer is injected into the air pouch. The pouch is gently massaged, the exudate immediately removed, and exudate volume recorded. The exudate is collected in a serum separator tube on an ice bath. The exudates are centrifuged (refrigerated) and an aliquot of the supernatant is stored in a labeled Eppendorf tube at −80° C.

Termination of Study: Animals are euthanized via CO₂ asphyxiation at the completion of the in-life portion of this study and carcasses are disposed of according to standard protocols.

Data analysis: The exudate samples are thawed to room temperature and assayed by ELISA for PGE2 (R&D Systems, Cat. KGE004B, lot 307711). Statistical significance of treatments on mean exudate volumes are determined by comparison of means for treatment groups with vehicle group. Mean cytokine concentrations and standard deviations are determined for each group. Statistical significance of treatments on cytokine concentrations are determined for each compound group compared to vehicle group. Statistical significance (p-value) is calculated vs control groups by Student's t-Test.

### Evaluation of COX-1 & COX-2 Activity In Vitro

The present invention includes compounds that are celecoxib conjugates, therefore they are evaluated for selective COX-1 or COX-2 inhibition. Assays for COX-1 and COX-2 activity in vitro are described in U.S. Pat. No. 5,760,068.

Preparation of Recombinant COX-1 and COX-2:
(1) A fragment containing the coding region of either human or murine COX-1 or COX-2 is cloned into a BamH1 site of a baculovirus transfer vector to generate transfer vectors for COX-I and COX-II.
(2) Recombinant baculoviruses are isolated by transfecting baculovirus transfer vector DNA into SF9 insect cells.
(3) Recombinant viruses are purified and high titer stocks of virus are prepared.
(4) SF9 insect cells are infected with the recombinant baculovirus stock. After 72 h the cells are centrifuged and the cell pellet homogenized. The homogenate is centrifuged and the supernatant is assayed for COX activity.

Assay for COX-1 and COX-2 Activity:
(1) COX activity is assayed as PGE2 formed/μg protein/time using an ELISA to detect the prostaglandin formed.
(2) Insect cell membranes containing the appropriate COX enzyme are incubated in buffer containing arachidonic acid.
(3) Compounds are pre-incubated with the enzyme for 10-20 minutes prior to the addition of arachidonic acid.
(4) Reaction between the arachidonic acid and the enzyme is stopped after ten minutes and the PGE2 formed is measured by standard ELISA technology.

### Assessment of Anti-Proliferative Activity

Anti-tumor growth potential of test compounds are evaluated in vitro using various human tumor cells, available from the American Type Culture Collection (ATCC), such as A549 lung tumor cells, DU145 prostate tumor cells, HT29 colon cancer cells, MIA PaCa-2 pancreatic cancer cells, MCF-7 (ER⁺) breast tumor cells and BEAS-2B cells (immortalized normal lung epithelial cells) as control (Clin. Cancer Res. 6, 2006-2011 (2000)). Test compound effect on cell proliferation is determined using the MTT based cell proliferation assay. MTT based cell proliferation assays are described in U.S. Pat. No. 8,143,237.

MTT [3-(4,5-dimethylthiazol-2-yl)-2,5-diphenyltetrazolium bromide] incorporation based cell proliferation assay is performed using the MTT cell proliferation assay kit (Roche Applied Sciences, Germany). The assay is carried out according to the instruction provided by the vendor. Briefly, equal numbers of cells are plated in 96-well flat-bottomed plates and are incubated with test compounds at various concentrations for a period of three days. Vehicle control culture wells receive an equal volume of vehicle solution. Thereafter, 0.5 mg/ml of MTT reagent is added to each well and the microplate is incubated further for 4 h at 37° C. in presence of 5% CO₂. Cells are then solubilized by adding solubilizing solution and allowed to incubate at 37° C. overnight. After complete solubilization of the formazan crystals, the absorbance is read at 540 nm in a microplate reader (BioRad, USA). The results (mean optical density (OD)±standard dethroughtion (SD)) obtained from quadruplicate wells are used to calculate the inhibition of cell proliferation (50% of inhibitory concentration, IC₅₀) of the test compounds.

### Suppression of Lung Cancer Cell Migration

Efficacy testing is done to evaluate test compound suppression of lung cancer cell migration, a model of metastasis. Methods to evaluate lung cancer cell migration are described in Mol. Med. Reports 3, 1007-1013 (2010).

Cell Culture: Human lung cancer cells A549 are obtained from American Type Culture Collection (ATCC, Manassas, Va.). Cells are incubated in RPMI-1640 medium supplemented with 10% fetal bovine serum (FBS) and penicillin/streptomycin (GibcoBRL, Grand Island, N.Y., USA).

### Monolayer Wound Healing Assay

Cell proliferation in confluent A549 monolayers is blocked by a 30 minute pre-incubation in the presence of mitomycin C (3 μg/ml). Test compounds, in cell culture buffer, are added to confluent monolayers 30 minutes before wound induction. A549 monolayers are subsequently scratched with a pipette tip. Wound areas are evaluated with phase contrast microscopy on an inverted microscope. Images of the same areas are obtained at intervals from zero to 96 h. Cell migration rate through wound healing is evaluated from the images using Paint.Net v.3.10 software. Cell migration is expressed as the fold change in the migration area, relative to untreated control cells at the same time period.

### Compound Formulations for Intravenous (IV), Oral Gavage (PO) or Intraperitoneal (IP) Administration

Compounds are formulated for administration using 25% hydroxypropyl-beta-cyclodextrin-PBS buffer (HBCD-PBS) at 1 mg/ml. HBCD-PBS is the preferred formulation media for compound administration. Additional formulation vehicles may also be used, including 2% Tween 80 in saline and 20% polyethylene glycol (PEG-300) in 0.9% sodium chloride in water.

### Determination of Maximum Tolerated Dose (MTD) of Test Compounds in Rats

In order to estimate the doses of test compounds for use in efficacy testing in animal models of cancer, the dosage at which adverse events occur is determined. Methods to determine MTD in rats are described in Mol. Cancer Ther. 5, 1530-1538 (2006).

In order to determine doses for efficacy studies, the maximum tolerated dose (MTD) is determined. Male F344 rats are fed various concentrations of test compounds for six weeks. MTD is determined based on the highest dose that causes a 10% loss in body weight without mortality or signs of toxicity. Body weights are recorded twice weekly. Animals are examined daily for signs of toxicity. At termination, animals are euthanized and organs dissected and examined.

### Compound Metabolism (PK) in Rats

The pharmacokinetics (PK) of compounds is tested by single dose IV administration to Sprague Dawley rats.

For each test compound, three (3) Sprague Dawley (CD® IGS) male rats are used. Animals are weighed and dosed individually by body weight on the day of treatment. Compounds are administered intravenously (IV), through surgically placed jugular catheters, at 10 mg/kg using 10 ml/kg volume per animal. Animals found in severe distress or a moribund condition are euthanized. Peripheral blood collections are done primarily through venipuncture of the tail or saphenous veins at various times (T=15 min, 30 min, 1 h, 2 h, 4 h, 8 h and 24 h). Whole blood samples are collected in an EDTA microtainer, processed to plasma by centrifugation and the plasma frozen at −80° C. Bioanalysis is done using LC/MS/MS methods using standard reverse phase HPLC and API 4000 triple quadrupole mass spectrometry. The amount of compound present is used to calculate PK parameters Cₘₐₓ, Tₘₐₓ and AUC.

### Compound Effects on Blood Pressure

COX-2 inhibitors have been shown to have adverse effects on blood pressure in vivo, the effect of the present compounds is evaluated for blood pressure effects in spontaneously hypertensive rats (SHR).

Thirty-two male, spontaneously hypertensive rats (SHR), 12-weeks old (four groups of eight) are used in this study. Initially, mean arterial blood pressure (MAP) is measured through tail-cuff daily, throughout the study. Animals undergo 2 days of blood pressure training and 1 day of baseline blood pressure measurements. Animals are weighed and dosed individually by body weight on the day of treatment. Compounds are administered orally (PO) or by intraperitoneal (IP) injection once on Day 1 at 10 mg/kg using 10 ml/kg volume per animal. Blood pressures are monitored for 6 days post-dose. A total of 7 time points are measured: Day 0 for baseline and Days 1, 2, 3, 4, 5 and 6 of the study. Animals found in severe distress or in a moribund condition are euthanized. Celecoxib is the positive control tested in these studies.

Anti-inflammatory Efficacy: Rat Carrageenan Foot Pad Edema: The compounds of the present invention are conjugates of celecoxib, therefore they are evaluated for efficacy in vivo in a model of inflammation. Methods to determine efficacy in rat carrageenan foot pad edema are described in U.S. Pat. No. 5,760,068.

Male Sprague Dawley rats are selected for equal average body weight per group. After fasting, with free access to water 16 h prior to test, animals are dosed orally (1 mL) with test compounds in a vehicle containing 0.5% methylcellulose and 0.025% surfactant. The control group is dosed with vehicle alone.

One hour after dosing, a subplantar injection of 0.1 mL of 1% solution of carrageenan/sterile 0.9% saline is administered in one foot, to all animals. The volume of the injected foot is measured using a displacement plethysmometer. Foot volume is measured again 3 h after carrageenan injection. The three hour foot volume measurement is compared between treated and control groups; the percent inhibition of edema is calculated.

### Anti-Inflammatory Efficacy—Rat Carrageenan-Induced Analgesia Test

The compounds of the present invention are conjugates of celecoxib, therefore they are evaluated for efficacy in vivo in a model of inflammatory analgesia. Methods to determine efficacy in rat carrageenan-induced analgesia test are described in U.S. Pat. No. 5,760,068.

Male Sprague Dawley rats are selected for equal average body weight per group. After fasting, with free access to water 16 h prior to test, animals are dosed orally (1 mL) with test compounds in vehicle containing 0.5% methylcellulose and 0.025% surfactant. Control groups are dosed with vehicle alone.

One hour after dosing, a subplantar injection of 0.1 mL of 1% solution of carrageenan/sterile 0.9% saline is administered in one foot, to all animals. Three hours after carrageenan injection, rats are placed in a plexiglass container with a high intensity lamp under the floor. After twenty minutes, thermal stimulation is begun on either the injected or the uninjected foot. Foot withdrawal is determined by a photoelectric cell. The time until foot withdrawal is measured and compared between treated and control groups. The percent inhibition of the hyperalgesic foot withdrawal is calculated.

### Tumor Growth Inhibition in Xenograft Mouse Model of NSCLC

Efficacy testing is done in animal models of cancer tumors. Methods to determine tumor growth inhibition in xenograft mouse models of NSCLC are described in Clin. Cancer Res. 7, 724-733 (2001).

Female HRLN nu/nu mice are injected subcutaneously with 1×10⁷ MV-522 cells in 0.1 ml of phosphate-buffered saline. Treatment is initiated when tumors measure 5x5 mm. Mice are weighed and tumors measured by calipers twice weekly. Animals are euthanized and tumors harvested and measured after 67 days or when animal dies. Drug efficacy is measured based on animal survival and tumor growth.

### Tumor Growth Inhibition in Xenograft Mouse Model of Colon Cancer

Efficacy testing is done in animal models of cancer tumors. Methods to determine tumor growth inhibition in xenograft mouse models of colon cancer are described in J. Drug Delivery 2011, 1-9 (Article ID 869027).

Female HRLN nu/nu mice are injected subcutaneously with 5×10⁷ HT-29 cells in 0.1 ml of phosphate-buffered saline. Treatment is initiated when tumors measure 5×5 mm. Mice are weighed and tumors measured by calipers twice weekly. Animals are euthanized and tumors harvested and measured after 67 days or when animal dies. Drug efficacy is measured based on animal survival and tumor growth.

### Growth Inhibition of Gallbladder Adenocarcinoma in Transgenic Mice

Efficacy testing is done in animal models of cancer tumors. Gallbladder adenocarcinoma in transgenic mice is described in Mol. Cancer Ther. 6, 1709-1717 (2007).

Homozygous BK5.ErbB-2 transgenic mice, that overexpress rat ErbB-2 and nontransgenic littermates receive a control AIN76A diet or an experimental diet containing the test compound for one month. The transgenic mice develop adenocarcinoma of the gallbladder with a 90% incidence. Ultrasound image analysis and histologic evaluation are used to determine compound effects on gall bladder tumor reversion to a milder phenotype and inhibition of tumor progression.

### Inhibition of Colon Cancer in Azomethane-Treated Rats

Efficacy testing is done in animal models of cancer tumors. Colon cancer in azomethane-treated rats is described in Mol. Cancer Ther. 5, 1530-1538 (2006).

Male F344 rats (Charles River Breeding Laboratories) are given test compounds blended into the diet. Efficacy of test compounds are determined following initiation of azoxymethane-induced colon cancer. Rats are randomly distributed by weight into various groups and housed in cages. Azomethane treated animals are injected subcutaneous (s.c.), twice weekly, at 15 mg/kg body weight. Vehicle-treated groups are injected with normal saline. Rats are placed on control diet or diets containing test compounds, two weeks after the second injection of azomethane or saline. Body weights are measured every two weeks until termination, 52 weeks after the last azoxymethane treatment. Organs are dissected and examined using a dissecting microscope.

Colon tumors with a diameter of >0.4 cm are fixed in 10% neutral buffered formalin for histopathologic evaluation. Test compounds are evaluated for effect on colonocyte proliferation. Proliferating cell nuclear antigen (PCNA) expression is determined by immunohistochemistry. Paraffin-embedded colons are sectioned and mounted on slides. PCNA antibody (PharMingen, San Diego, Calif.), at a 1:200 dilution, is added for 1 hour. Sections are washed, then incubated with secondary anti-rabbit IgG (30 minutes). Following washing, avidin biotin-complex reagent (Vector Laboratories, Burlingame, Calif.) is added. Sections are washed and 3,3″-diaminobenzidine is added and sections are counterstained with hematoxylin. Proliferation index is calculated based on the number of positive cells (brown nucleus) per crypt.

All mentioned documents are incorporated by reference as if herein written. When introducing elements of the present invention or the exemplary embodiment(s) thereof, the articles “a,” “an,” “the”, and “said” are intended to mean that there are one or more of the elements. The terms “comprising”, “including”, and “having” are intended to be inclusive and mean that there may be additional elements other than the listed elements. Although this invention has been described with respect to specific embodiments, the details of these embodiments are not to be construed as limitations.

What is claimed is:

1. A compound, or pharmaceutically acceptable salt or solvate of a compound or salt of Formula (I): 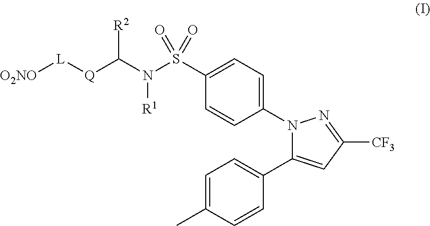
wherein:
R¹ is selected from the group consisting of acyl, arylcarbonyl, heteroarylcarbonyl, carboxyalkylenecarbonyl, and alkyl-O-carbonylalkylenecarbonyl;
R² is selected from the group consisting of H, alkyl, cycloalkyl, aryl, aralkyl, and heterocyclyl;
-Q- is selected from the group consisting of O, 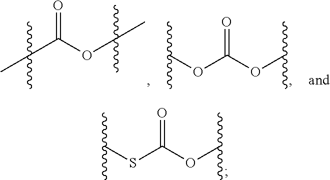 [SMILES: *OC(*)=O ; *OC(=O)O* ; *OC(=O)S*]
-L- is C₁₋₈ alkylene, wherein one, two, or three —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂;
R³ is independently selected from the group consisting of H, alkyl, aryl, aralkyl, heterocyclyl, halogen, carboxy, carboxyalkylene, acyl, and nitrooxy C₁₋₃ alkylene; and
R⁴ is selected from the group consisting of alkyl, aryl, aralkyl, heterocyclyl, carboxyalkylene, and acyl.

2. Compound of claim 1, of Formula (II) 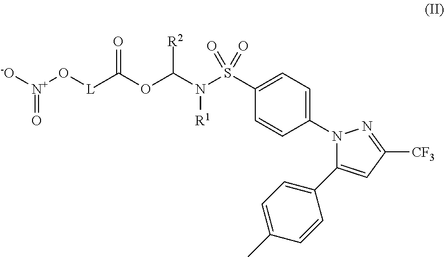
wherein:
R¹ is acyl;
R² is selected from the group consisting of H, alkyl, and cycloalkyl;
-L- is C₁₋₆ alkylene, wherein one or two —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂;
R³ is independently selected from the group consisting of H, alkyl, aryl, aralkyl, heterocyclyl, halogen, carboxy, carboxyalkylene, acyl, and nitrooxy C₁₋₃ alkylene; and
R⁴ is selected from the group consisting of alkyl, aryl, aralkyl, heterocyclyl, carboxyalkylene, and acyl.

3. Compound of claim 2, wherein:
R² is selected from the group consisting of H and alkyl;
-L- is C₁₋₆ alkylene, wherein one —CH₂— radical may be optionally replaced with —CH(R³)—; and
R³ is selected from the group consisting of H, alkyl, and aryl.

4. Compound of claim 3, wherein:
R¹ is acetyl;
R² is H or methyl;
-L- is C₁₄ alkylene, wherein one —CH₂— radical may be optionally replaced with —CH(R³)—; and
R³ is selected from the group consisting of H, methyl, or phenyl.

5. Compound of claim 4, selected from the group consisting of:
(N-((4-(5-(p-tolyl)-3-(trifluoromethyl)-1H-pyrazol-1-yl)phenyl)sulfonyl)acetamido)methyl 2-(nitrooxy)acetate;
1-(N-((4- (5- (p-tolyl)-3-(trifluoromethyl)-1H-pyrazol-1-yl)phenyl)sulfonyl)acetamido)ethyl 2-(nitrooxy)acetate;
(N-((4-(5-(p-tolyl)-3-(trifluoromethyl)-1H-pyrazol-1-yl)phenyl)sulfonyl)acetamido)methyl 2-(nitrooxy)propanoate;
(N-((4-(5-(p-tolyl)-3-(trifluoromethyl)-1H-pyrazol-1-yl)phenyl)sulfonyl)acetamido)methyl 3-(nitrooxy)propanoate
(N-((4-(5-(p-tolyl)-3-(trifluoromethyl)-1H-pyrazol-1-yl)phenyl)sulfonyl)acetamido)methyl 4-(nitrooxy)butanoate;
(N-((4-(5-(p-tolyl)-3-(trifluoromethyl)-1H-pyrazol-1-yl)phenyl)sulfonyl)acetamido)methyl 5-(nitrooxy)pentanoate; and
(N-((4-(5-(p-tolyl)-3-(trifluoromethyl)-1H-pyrazol-1-yl)phenyl)sulfonyl)acetamido)methyl 2-(nitrooxy)-2-phenylacetate.

6. Compound of claim 1, of Formula (III) 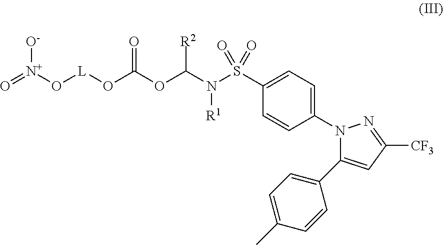
wherein:
R¹ is acyl;
R² is selected from the group consisting of H, alkyl, and cycloalkyl;
-L- is C₂₋₆ alkylene, wherein one or two —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂;
R³ is independently selected from the group consisting of H, alkyl, aryl, aralkyl, heterocyclyl, halogen, carboxy, carboxyalkylene, acyl, and nitrooxy C₁₋₃ alkylene; and
R⁴ is selected from the group consisting of alkyl, aryl, aralkyl, heterocyclyl, carboxyalkylene, and acyl.

7. Compound of claim 6, wherein:
R² is selected from the group consisting of H and alkyl;
-L- is C₂₋₆ alkylene, wherein one —CH₂— radical may be optionally replaced with —CH(R³)—; and
R³ is selected from the group consisting of H, alkyl, aryl, and nitrooxy C₁₋₃ alkylene.

8. Compound of claim 7, wherein:
R¹ is acetyl;
R² is H or methyl;
-L- is C₂₄ alkylene, wherein one —CH₂— radical may be optionally replaced with —CH(R³)—; and
R³ is selected from the group consisting of H, methyl, or nitrooxymethylene.

9. Compound of claim 8, selected from the group consisting of:
2-(nitrooxy)ethyl ((N-((4-(5-(p-tolyl)-3-(trifluoromethyl)-1H-pyrazol-1-yl)phenyl)sulfonyl)acetamido)methyl)carbonate;
1-(nitrooxy)propan-2-yl 4N-((4-(5-(p-tolyl)-3-(trifluoromethyl)-1H-pyrazol-1-yl)phenyl)sulfonyl)acetamido)methyl)carbonate;
2-(nitrooxy)propyl ((N-((4-(5-(p-tolyl)-3-(trifluoromethyl)-1H-pyrazol-1-yl)phenyl)sulfonyl)acetamido)methyl)carbonate;
3-(nitrooxy)propyl ((N-((4-(5-(p-tolyl)-3-(trifluoromethyl)-1H-pyrazol-1-yl)phenyl)sulfonyl)acetamido)methyl)carbonate;
2,3-bis(nitrooxy)propyl ((N-((4-(5-(p-tolyl)-3-(trifluoromethyl)-1H-pyrazol-1-yl)phenyl)sulfonyl)acetamido)methyl)carbonate;
1,3-bis(nitrooxy)propan-2-yl ((N-((4-(5-(p-tolyl)-3-(trifluoromethyl)-1H-pyrazol-1-yl)phenyl)sulfonyl)acetamido)methyl)carbonate; and
4-(nitrooxy)butyl ((N-((4-(5-(p-tolyl)-3-(trifluoromethyl)-1H-pyrazol-1-yl)phenyl)sulfonyl)acetamido)methyl)carbonate.

10. Compound of claim 1, of Formula (IV) 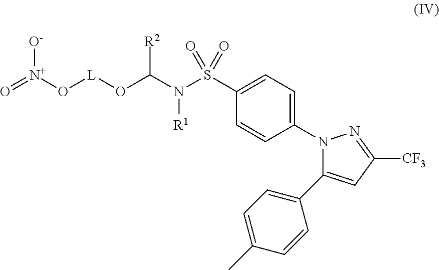
wherein:
R¹ is acyl;
R² is selected from the group consisting of H, alkyl, and cycloalkyl;
-L- is C₂₋₆ alkylene, wherein one or two —CH₂— radicals may be replaced with a radical independently selected from the group consisting of CH(R³) and C(R³)₂, or -L- is selected from the group consisting of CH₂CH₂OCH₂CH₂, CH₂CH₂SCH₂CH₂, and CH₂CH₂N(R⁴)CH₂CH₂;
R³ is independently selected from the group consisting of H, alkyl, aryl, aralkyl, heterocyclyl, halogen, carboxy, carboxyalkylene, acyl, and nitrooxy C₁₋₃ alkylene; and
R⁴ is selected from the group consisting of alkyl, aryl, aralkyl, heterocyclyl, carboxyalkylene, and acyl.

11. Compound of claim 10, wherein:
R² is selected from the group consisting of H and alkyl;
-L- is C ₂₋₆ alkylene, wherein one —CH₂— radical may be optionally replaced with —CH(R³)—; and
R³ is selected from the group consisting of H, alkyl, and nitrooxy C₁₋₃ alkylene.

12. Compound of claim 11, wherein:
R¹ is acetyl;
R² is H or methyl;
-L- is C₂₄ alkylene; and
R³ is H.

13. Compound of claim 12, selected from the group consisting of:
2-((N-((4-((5-(p-tolyl)-3-(trifluoromethyl)-1H-pyrazol-1-yl)phenyl)sulfonyl)acetamido)methoxy)ethyl nitrate; and
2-((N-((4-(5-(p-tolyl)-3-(trifluoromethyl)-1H-pyrazol-1-yl)phenyl)sulfonyl)acetamido)methoxy)butyl nitrate.

14. A pharmaceutical composition comprising a therapeutically effective amount of a compound of claim 1, and a pharmaceutically acceptable carrier.

15. The pharmaceutical composition of claim 14, further comprising a therapeutically effective amount of an active pharmaceutical ingredient selected from the group consisting of anti-inflammatory drugs, cytostatic drugs, cytotoxic drugs, anti-proliferative agents, and angiogenesis inhibitors.

16. A method for treating or preventing a disease condition comprising administering to a mammalian subject in need thereof a therapeutically effective amount of a compound of claim 1, wherein the disease condition is selected from the group consisting of cancer, actinic keratosis, cystic fibrosis, and acne.

17. The method of claim 16, wherein the disease condition is selected from the group consisting of non-small cell lung cancer, skin cancer, liver cancer, colorectal cancer (including metastatic colorectal cancer, and FAP), glioblastoma (and other CNS related cancers), squamous cell cancer, bladder cancer, breast cancer, biliary tract cancer, cervical cancer, prostate cancer, small cell lung cancer, ovarian cancer, pancreatic cancer, and gastrointestinal cancer.

18. The method of claim 17, wherein the condition is non-small cell lung cancer or colorectal cancer.

19. A method for healing a wound comprising administering to a mammalian subject in need thereof a therapeutically effective amount of a compound of claim 1.

20. A method for treating or preventing a disease condition comprising administering to a mammalian subject in need thereof a therapeutically effective amount of a compound of claim 1, wherein the disease condition has COX-2 over-expression.
